(19) United States
(12) Patent Application Publication
(10) Pub. No.: US 2026/0360008 A1
(43) Pub. Date: Nov. 12, 2026

(54) METHOD OF TRACING AN ELONGATE OBJECT IN IMAGE DATA AND IMAGE PROCESSING SYSTEM

(71) Applicant: MAX-PLANCK-GESELLSCHAFT ZUR FÖRDERUNG DER WISSENSCHAFTEN E.V., München (DE)

(72) Inventors: Martin SCHMIDT, Mannheim (DE); Alessandro MOTTA, Luzern (CH); Moritz HELMSTAEDTER, Königstein im Taunus (DE)

(21) Appl. No.: 19/100,295

(22) PCT Filed: Aug. 4, 2023
(86) PCT No.: PCT/EP2023/071656

§ 371 Date: Jan. 31, 2025

(30) Foreign Application Priority Data
Aug. 4, 2022 (EP) 22188663.3

(51) Int. Cl. G06T 7/73; A61B 34/20; A61M 25/01; G06T 7/00; G06V 10/764; G06V 10/774; G06V 10/82; G16H 30/40

(52) U.S. Cl. CPC G06T 7/74; A61B 34/20; G06T 7/0014; G06V 10/764; G06V 10/774; G06V 10/82; G16H 30/40; A61B 2034/2065; A61M 2025/0166; G06T 2200/04; G06T 2207/20081; G06T 2207/20084; G06T 2207/30101; G06V 2201/03

(57) ABSTRACT

Methods and systems for tracing an elongate object in image data are disclosed. The methods and systems may be operative for, e.g., neurite tracing. An artificial intelligence model is used to infer a steering prediction. The steering prediction is processed to determine a next one of a series of points (60) on a center line (50) of the elongate object.

### TECHNICAL FIELD

The invention relates to image processing methods and systems. Embodiments of the invention relate to methods and systems for tracing an elongate object, such as a neurite, in image data representative of a three-dimensional tissue volume.

### BACKGROUND

Dense mapping of neuronal wires and their synaptic connections for enhanced understanding of computation in the brain is a major goal of high-resolution connectomics. While acquisition of mm³-sized 3-dimensional electron microscopy (3D-EM) datasets is feasible, axon and spine neck reconstruction remain a considerable challenge.

Human annotations of 3D image data can provide low error rates but are frequently prohibitively time-consuming. Thus, there is a need for computer-aided or computer-implemented techniques that are capable of tracing elongate objects in image data representing a three-dimensional (3D) tissue volume. Neurite reconstruction by means of a sparse trace along their center line (“skeletonization”) yielded an about 50-fold increase in annotation speed (see, e.g., M. Helmstaedter et al. 2011. “High-Accuracy Neurite 593 Reconstruction for High-Throughput Neuroanatomy.” Nature Neuroscience 14 (8):594 1081-88). This approach is at the basis of recent large-scale reconstructions (see, e.g., K. Eichler et al. 2017. “The Complete Connectome of a 579 Learning and Memory Centre in an Insect Brain.” Nature 548 (7666): 175-82). Skeleton reconstructions have been combined with automatically obtained segmentations to yield volume reconstructions (see, e.g., Helmstaedter et al. 2013. “Connectomic Reconstruction of the Inner Plexiform Layer in the Mouse Retina.” Nature 500 (7461): 168-74; Dorkenwald et al. 2017. “Automated Synaptic Connectivity Inference for Volume Electron Microscopy.” Nature Methods 14 (4): 435-42; and Staffler et al. 2017. “SynEM, Automated Synapse Detection for Connectomics.” ELife 6 (July): e26414).

An additional speed-up was achieved by skeletonizing neurites using a user interface in which the annotator is presented with an ego-centric view of the 3D-EM volume (“flight mode”) (see, e.g., Boergens et al. 2017. “WebKnossos: Efficient Online 3D Data Annotation for Connectomics.” Nature Methods 14 (7): 691-94.). Other approaches aim at reducing the number of locations requiring human annotations by automatic reconstruction of neurites and subsequent detection of reconstruction errors (S. M. Plaza. 2016. “Focused Proofreading to Reconstruct Neural Connectomes from EM Images at Scale.” In Deep Learning and Data Labeling for Medical Applications, 249-58. Springer, Cham.; Y. Meirovitch et al. 2016. “A Multi-Pass Approach to Large620 Scale Connectomics.” ArXiv E-Prints, December. http://arxiv.org/abs/1612.02120; D. Rolnick et al. 2017. “Morphological Error Detection in 3D Segmentations.” ArXiv E-Print. https://arxiv.org/pdf/1705.10882.pdf; J. Zung et al. 2017. “An Error Detection and Correction Framework for Connectomics.” NIPS, 6818-29; K. Dmitriev et al. 2018. “Efficient Correction for EM Connectomics with Skeletal Representation.” In British Machine Vision Conference (BMVC). http://bmvc2018.org/contents/papers/0064.pdf; and A. Motta et al. 2019. “Dense Connectomic Reconstruction in Layer 4 of the Somatosensory Cortex.” Science. Vol 366. Issue 6469. p. eaay3134).

Additional exemplary techniques for tracing an object are disclosed in: Jelmer M. Wolrterink et al.: “Coronary artery centerline extraction in cardiac CT angiography using a CNN-based orientation classifier”, MEDICAL IMAGE ANALYSIS, OXFORD UNIVERSITY PRESS, OXOFRD, GB, vol. 51, 22 Oct. 2018 (2018-10-22), pages 46-60, ISSN: 1361-8415,DOI: 10.1016/J.MEDIA.2018.10.005; Rouchen Gao et al.: “Joint Coronary Centerline Extraction And Lumen Segmentation From Ccta Using Cnntracker And Vascular Graph Convolutional Network”, 2021 IEEE 18TH INTERNATIONAL SYMPOSIUM ON BIOMEDICAL IMAGING (ISBI), IEEE, 13 Apr. 2021 (2021-04-13), pages 1897-1901, DOI: 10.1109/ISB|4821120219433764; and Tianhong Dai et al.: “Deep Reinforcement Learning for Subpixel Neural Tracking”, Proceedings of Machine Learning Research, 2019, pages 130-150, http://proceedings.mlr.press/v102/dai19a/dai19a.pdf.

### SUMMARY

There remains a need for enhanced techniques of processing images. There remains in particular a need for techniques that are capable of tracing elongate objects, such as neurites, in a computationally efficient manner and/or with a low error rate.

According to embodiments of the invention, methods and systems as recited in the independent claims are provided. The dependent claims define preferred embodiments.

A method of tracing an elongate object in image data, the image data being biological or medical image data, may comprise determining, by an image processing system, positions of a series of points along the elongate object in three-dimensional, 3D, space to trace the elongate object in 3D in the image data. The series of points may be determined such that they are located along an approximation of a center line of the elongate object.

Determining the positions of the series of points may comprise an iterative process.

The iterative process may comprise inferring, using an artificial intelligence (AI) model, a steering prediction that is dependent on (and which may be indicative of) a direction along which the elongate object (e.g., a center line or approximation of the center line) extends from a previously determined point along the elongate object (e.g., along the center line or an approximation of the center line), the AI model comprising an input layer operative to receive an AI model input that is based on pixel or voxel values of the image data and an output layer operative to provide, in each iteration of the iterative process, an AI model output that is or indicates the steering prediction.

The iterative process may comprise computing, using at least the steering prediction, a next point of the series of points, the next point following the previously determined point in the series of points.

The iterative process may comprise iteratively repeating the inferring and computing steps until a termination criterion is fulfilled, to thereby determine the series of points.

According to a preferred embodiment, the AI model may comprise a convolutional neural network (CNN) comprising the input layer, a plurality of convolutional layers, a dropout layer or several dropout layers, and the output layer, or a recurrent neural network (RNN), wherein the AI model input that is input to the input layer in an iteration of the iterative process represent a sub-volume of a tissue volume represented by the image data, wherein the AI model input comprises pixel or voxel values representing the sub-volume of the image data or is obtained by multivariate interpolation of pixel or voxel values representing the sub-volume of the image data, wherein an orientation of the sub-volume is dependent on the steering prediction determined in the preceding iteration of the iterative process.

According to a preferred embodiment, the steering prediction may be dependent on a second derivative of the elongate object, e.g., of a center line of the elongate object.

According to another preferred embodiment, the AI model may be a convolutional neural network (CNN) comprising the input layer, a plurality of convolutional layers, a dropout layer or several dropout layers, and the output layer, or a recurrent neural network (RNN), wherein the AI model input that is input to the input layer in an iteration of the iterative process represent a sub-volume of a tissue volume represented by the image data, the steering prediction being curvature values relative to a reference frame given in relation to the sub-volume such that it yields a position offset for determining in an iterative manner a series of points located on a center line of the elongate object, and in each iteration following an immediately preceding iteration, the respective subvolume that is processed is determined such as to have a rotational orientation that is determined by the direction of extension of the elongate object as determined from the steering prediction obtained in the immediately preceding iteration.

The method may comprise outputting a result of the image processing via a graphical user interface or via a data interface.

Determining the next point of the series of points may comprise integrating the steering prediction.

The steering prediction may be dependent on a second derivative of the center line of the elongate object.

The AI model input that is input to the input layer in an iteration of the iterative process may represent a sub-volume of a tissue volume represented by the image data.

The sub-volume may be dependent on a preceding steering prediction determined in a preceding iteration of the iterative process and/or the point that has been determined in the preceding iteration of the iterative process.

An orientation of the sub-volume may be dependent on the preceding steering prediction and/or may be dependent on uncertainties of predictions of the AI model over multiple orientations.

The orientation of the sub-volume may depend on a direction along which the elongate object extends at or within the sub-volume.

The orientation of the sub-volume may be approximately aligned with a direction along which the elongate object extends at or within the sub-volume.

A center of the sub-volume may be shifted relative to the previously determined point by an offset, further optionally wherein the offset is dependent on the steering prediction determined in the preceding iteration.

The method may comprise multivariate interpolation (such as averaging) the pixel or voxel values of the image data to determine the AI model input.

Multivariate interpolation may comprise weighted averaging of the pixel or voxel values to generate the AI model input as interpolated pixel or voxel values on a grid that is tilted and/or offset relative to a pixel or voxel grid of the image data.

Multivariate interpolation may comprise a trilinear projection.

The method may comprise performing an initialization of the iterative procedure.

The initialization may be dependent on whether an initial orientation is available a priori.

When the initial orientation is not available, the method may comprise determining the initial orientation for use in an initial iteration of the iterative process.

Determining the initial orientation may comprise determining uncertainty estimates for the AI model predictions for various orientations and choosing the orientation from among the various orientations for which the uncertainty estimate is minimum.

The uncertainty estimates may be determined using Monte-Carlo dropout.

The AI model may comprise a convolutional neural network (CNN).

The AI model may comprise a recurrent neural network (RNN).

The AI model may comprise the input layer, a plurality of convolutional layers, a dropout layer or several dropout layers, and the output layer.

The output layer may be a linear layer.

The plurality of convolutional layers may comprise one or several strided convolutional layers.

The AI model may be or may comprise a fully convolutional network.

The AI model may comprise one or several of pooling layers, attention modules, self-attention modules, atrous convolutions, normalization layers, batch normalization layers, transformers, recurrent layers.

The AI model may comprise one or several nonlinearities.

The AI model may comprise exponential linear units (ELU).

The AI model may comprise rectified linear units (ReLU).

The AI model may comprise Scaled Exponential Linear units (SELU).

The AI model may comprise Gaussian Error Linear Unit (GELU).

The image data may be representative of a volumetric representation of tissue.

The image data may comprise a three-dimensional (3D) image or a set of two-dimensional (2D) images.

The method further may comprise acquiring the image data.

The image data may be acquired using electron microscopy.

The image data may be acquired using optical microscopy, e.g., light microscopy.

The image data may be acquired using positron emission tomography (PET), magnetic resonance (MR) imaging, X-ray tomography, or another medical imaging modality.

The elongate object may be a neurite.

The elongate object may be a blood vessel.

The elongate object may have a surface that extends around the center line in a locally cylindrical manner.

The method may be used for axon tracing.

The elongate object may be an axon.

The elongate object may be a spine neck.

The method may be performed multiple times in parallel and/or sequentially to trace a plurality of axons and/or dendrites.

The method may comprise training the AI model.

The AI model may be trained using supervised learning.

Training the AI model may comprise gradient-based updates of AI model parameters.

Training the AI model may comprise training the AI model to comply with a flight policy that causes the iterative process to converge to the elongate object (e.g., along the center line or an approximation of the center line) when starting from an initial location in the image data that is off the center line.

A convergence distance of the flight policy may determine how rapidly the iterative process converges back to the center line.

The convergence distance may be a dynamic parameter that is varied during AI model training and/or inference.

The convergence distance may be set as a function of a distance from a physiological boundary during AI model training.

The convergence distance may be a monotonously increasing function of the distance from the physiological boundary.

The method may comprise outputting one or several of a visual representation of the series of points or of the center line; a machine-readable representation of the series of points or of the center line; and a control signal that is dependent on the series of points located along the center line; and uncertainties of the predicted control signal; and a representation of which stop criterion/criteria ended the iterative process.

The outputting may be performed via a user interface, such as a graphical user interface, or via a data interface. A medical imaging method or a medical image processing method comprises receiving medical image data representing a tissue volume and comprising a 3D image or a set of 2D images of tissue comprising neural tissue, performing the image processing method to trace an elongate object in the medical image data, and outputting one or several of a visual representation of the series of points or of the center line; a machine-readable representation of the series of points or of the center line; a control signal that is dependent on the series of points located along the center line; a machine learning (ML) model that has an architecture that depends on the series of points located along the center line.

A connectomics method comprises performing the method of tracing an elongate object.

A method of analyzing a bioptic sample may comprise acquiring image data of the bioptic sample, processing the image data using the method of processing image data, and outputting a result of the image data processing via an interface.

The method of analyzing the bioptic sample may be performed to determine part of a connectome.

The bioptic sample is a sample that is not being implanted into an animal or human body.

A method of generating a signal processing logic for controlling a physical asset may comprise performing the method of processing image data, using, by at least one computing system, the output of the elongate object tracing method to select or generate a machine learning, ML, model architecture, train an ML model having the ML model architecture, deploy the trained ML model as the signal processing logic to a control device for execution.

An image processing system for tracing an elongate object in image data may comprise an interface operative to receive the image data, the image data being biological or medical image data.

The image processing system may comprise at least one processing device operative to determine positions of a series of points along an elongate object (e.g., along the center line or an approximation of the center line) in three-dimensional, 3D, space to trace the elongate object in 3D in the image data, wherein determining the positions of the series of points may comprise an iterative process.

The iterative process may comprise inferring, using an artificial intelligence (AI) model, a steering prediction that is dependent on (and which may be indicative of) a direction along which the elongate object extends from a previously determined point along the elongate object, the AI model comprising an input layer operative to receive an AI model input that is based on pixel or voxel values of the image data and an output layer operative to provide, in each iteration of the iterative process, an AI model output that is or indicates the steering prediction.

The iterative process may comprise computing, using the steering prediction, a next point of the series of points, the next point following the previously determined point in the series of points.

The iterative process may comprise iteratively repeating the inferring and computing steps until a termination criterion is fulfilled, to thereby determine the series of points.

According to a preferred embodiment, the image processing system may be operative such that the AI model may comprise a convolutional neural network (CNN) comprising the input layer, a plurality of convolutional layers, a dropout layer or several dropout layers, and the output layer, or a recurrent neural network (RNN), wherein the AI model input that is input to the input layer in an iteration of the iterative process represent a sub-volume of a tissue volume represented by the image data, wherein the AI model input comprises pixel or voxel values representing the sub-volume of the image data or is obtained by multivariate interpolation of pixel or voxel values representing the sub-volume of the image data, wherein an orientation of the sub-volume is dependent on the steering prediction determined in the preceding iteration of the iterative process.

According to a preferred embodiment, the steering prediction may be dependent on a second derivative of the elongate object, e.g., of a center line of the elongate object.

According to another preferred embodiment, the image processing system may be operative such that the AI model may be a convolutional neural network (CNN) comprising the input layer, a plurality of convolutional layers, a dropout layer or several dropout layers, and the output layer, or a recurrent neural network (RNN), wherein the AI model input that is input to the input layer in an iteration of the iterative process represent a sub-volume of a tissue volume represented by the image data, the steering prediction being curvature values relative to a reference frame given in relation to the sub-volume such that it yields a position offset for determining in an iterative manner a series of points located on a center line of the elongate object, and in each iteration following an immediately preceding iteration, the respective subvolume that is processed is determined such as to have a rotational orientation that is determined by the direction of extension of the elongate object as determined from the steering prediction obtained in the immediately preceding iteration.

The image processing system may be operative to perform the method according to an embodiment.

The image processing system may comprise an image acquisition system operative to capture the image data.

The image acquisition system may be operative to perform 3D electron microscopy or light microscopy.

The image processing system may be operative to output a result of the image processing via a graphical user interface or via a data interface.

The image processing system may be operative such that determining the next point of the series of points may comprise integrating the steering prediction.

The image processing system may be operative such that the AI model input that is input to the input layer in an iteration of the iterative process may represent a sub-volume of a tissue volume represented by the image data.

The image processing system may be operative such that the sub-volume may be dependent on a preceding steering prediction determined in a preceding iteration of the iterative process and/or the point that has been determined in the preceding iteration of the iterative process.

The image processing system may be operative such that an orientation of the sub-volume may be dependent on the preceding steering prediction.

The image processing system may be operative such that the orientation of the sub-volume may depend on a direction along which the elongate object extends at or within the sub-volume.

The image processing system may be operative such that the orientation of the sub-volume may be approximately aligned with a direction along which the elongate object extends at or within the sub-volume.

The image processing system may be operative such that center of the sub-volume may be shifted relative to the previously determined point by an offset.

The image processing system may be operative such that the offset may be dependent on the steering prediction determined in the preceding iteration.

The image processing system may be for a multivariate interpolation (such as averaging) the pixel or voxel values of the image data to determine the AI model input.

The image processing system may be operative such that the multivariate interpolation may comprise weighted averaging of the pixel or voxel values to generate the AI model input as interpolated pixel or voxel values on a grid that is tilted and/or offset relative to a pixel or voxel grid of the image data.

The image processing system may be operative to perform projections to determine the pixel or voxel values on a grid that is tilted and/or offset relative to a pixel or voxel grid of the image data.

The image processing system may be operative to perform an initialization of the iterative procedure.

The image processing system may be operative such that the initialization may be dependent on whether an initial steering prediction is available a priori.

The image processing system may be operative such that, when the initial orientation is not available, the image processing system determines the initial orientation for use in an initial iteration of the iterative process.

The image processing system may be operative such that determining the initial orientation may comprise determining uncertainty estimates for various orientations and choosing the orientation from among the various orientations for which the uncertainty estimate is minimum.

The image processing system may be operative such that the uncertainty estimates may be determined using Monte-Carlo dropout.

The AI model may comprise a convolutional neural network (CNN).

The AI model may comprise a recurrent neural network (RNN).

The AI model may comprise the input layer, a plurality of convolutional layers, a dropout layer or several dropout layers, and the output layer.

The output layer may be a linear layer.

The plurality of convolutional layers may comprise one or several strided convolutional layers.

The AI model may be or may comprise a fully convolutional network.

The AI model may comprise one or several of pooling layers, attention modules, self-attention modules, atrous convolutions, normalization layers, batch normalization layers, transformers, recurrent layers.

The AI model may comprise exponential linear units (ELU).

The AI model may comprise rectified linear units (ReLU).

The AI model may comprise Scaled Exponential Linear units (SELU).

The AI model may comprise Gaussian Error Linear Unit (GELU).

The image data may be representative of a volumetric representation of tissue.

The image data may comprise a 3D image or a set of 2D images.

The image processing system may be operative to acquire the image data.

The image data may be acquired using electron microscopy.

The image data may be acquired using optical microscopy, e.g., light microscopy.

The image data may be acquired using positron emission tomography (PET), magnetic resonance (MR) imaging, or another medical imaging modality.

The elongate object may be a neurite.

The elongate object may be a blood vessel.

The elongate object may have a surface that extends around the center line in a locally cylindrical manner.

The image processing system may be operative to train the AI model.

The AI model may be trained using supervised learning.

The image processing system may be operative such that training the AI model may comprise gradient-based updates of AI model parameters.

The image processing system may be operative such that training the AI model may comprise training the AI model to comply with a flight policy that causes the iterative process to converge to the center line when starting from an initial location in the image data that is off the center line.

The image processing system may be operative such that a convergence distance of the flight policy may determine how rapidly the iterative process converges back to the center line.

The image processing system may be operative such that the convergence distance may be a dynamic parameter that is varied during AI model training.

The image processing system may be operative such that the convergence distance may be set as a function of a distance from a physiological boundary during AI model training.

The image processing system may be operative such that the convergence distance may be a monotonously increasing function of the distance from the physiological boundary.

The image processing system may be operative to output one or several of a visual representation of the series of points or of the center line; a machine-readable representation of the series of points or of the center line; and a control signal that is dependent on the series of points located along the center line.

The image processing system may comprise a user interface, such as a graphical user interface, or a data interface to perform the outputting.

A training method of training an AI model is also disclosed, the AI model comprising an input layer operative to receive an AI model input that is based on pixel or voxel values of image data representing a tissue volume and an output layer operative to provide an AI model output that is or indicates a steering prediction, the steering prediction being dependent on (and may be indicative of) a direction along which an elongate object (e.g., along the center line or an approximation of the center line) extends.

The training method may comprise training the AI model with an objective of predicting the direction along which the center line of an elongate object extends.

The training method may comprise training the AI model with an objective of outputting a derivative (e.g., a first order, second order, or higher order derivative) of the direction along which the center line of an elongate object extends.

The training method may be a gradient-based supervised learning method.

In the training method, the AI model may be trained using supervised learning.

The training method may comprise gradient-based updates of AI model parameters.

The training method may comprise training the AI model to comply with a flight policy that causes the iterative process to converge to the center line when starting from or reaching a location in the image data that is off the center line.

A convergence distance of the flight policy may determine how rapidly the iterative process converges back to the center line.

The convergence distance may be a dynamic parameter that is varied during AI model training and/or inference.

The convergence distance may be dependent on a distance from an obstacle.

The obstacle may be a physiological boundary, such as a cell boundary.

The convergence distance may be set as a function of a distance from a physiological boundary during AI model training.

The convergence distance may be a monotonously increasing function of the distance from the physiological boundary.

An AI model input that is input to the input layer may represent a sub-volume of a tissue volume represented by training data. The training method may comprise a multivariate interpolation of the pixel or voxel values of the image data to determine the AI model input.

The multivariate interpolation may comprise weighted averaging of the pixel or voxel values to generate the AI model input as interpolated pixel or voxel values on a grid that is tilted and/or offset relative to a pixel or voxel grid of the image data.

In the training method, the AI model may comprise a convolutional neural network (CNN).

In the training method, the AI model may comprise a recurrent neural network (RNN).

In the training method, the AI model may comprise the input layer, a plurality of convolutional layers, a dropout layer or several dropout layers, and the output layer.

In the training method, the output layer may be a linear layer.

In the training method, the plurality of convolutional layers may comprise one or several strided convolutional layers.

In the training method, the AI model may be or may comprise a fully convolutional network.

In the training method, the AI model may comprise one or several of pooling layers, attention modules, self-attention modules, atrous convolutions, normalization layers, batch normalization layers, transformers, recurrent layers.

In the training method, the AI model may comprise one or several nonlinearities.

In the training method, the AI model may comprise exponential linear units (ELU).

In the training method, the AI model may comprise rectified linear units (ReLU).

In the training method, the AI model may comprise Scaled Exponential Linear units (SELU).

In the training method, the AI model may comprise Gaussian Error Linear Unit (GELU).

In the training method, the image data may be representative of a volumetric representation of tissue.

In the training method, the image data may comprise a 3D image or a set of 2D images.

In the training method, the image data may be electron microscopy (EM) images.

In the training method, the image data may be 3D EM images.

In the training method, the image data may be X-ray tomography images.

In the training method, the image data may be optical microscopy, e.g., light microscopy data.

In the training method, the image data may be positron emission tomography (PET), magnetic resonance (MR) imaging, or other medical image data.

In the training method, the elongate object on which the AI model is trained may be a neurite.

In the training method, the elongate object on which the AI model is trained may be a blood vessel.

In the training method, the elongate object on which the AI model is trained may have a surface that extends around the center line in a locally cylindrical manner.

According to another embodiment, there is provided a method of tracing an object in image data. The method comprises determining a series of points along the object to trace the object in the image data.

Determining a series of points may comprise an iterative process. The iterative process may comprise:
inferring, using an artificial intelligence (AI) model, a steering prediction, the AI model comprising an input layer operative to receive an AI model input that is based on pixel or voxel values of the image data and an output layer operative to provide, in each iteration of the iterative process, an AI model output that is or indicates the steering prediction;
computing, using at least the steering prediction, a next point of the series of points, the next point following the previously determined point in the series of points; and
iteratively repeating the inferring and computing steps until a termination criterion is fulfilled.

The image data may be N-dimensional image data, with N being an integer. N may be at least 2, at least 3, or at least 4.

The object may be traced in N-D dimensions, where D is an integer equal to or greater than zero and less than N.

One of the dimensions of the image data may correspond to a time axis.

Determining the next point of the series of points may comprise integrating the steering prediction.

Integrating the steering prediction may comprise integrating curvatures in a Bishop reference frame.

The steering prediction is related to a second derivative of the center line of the object.

The AI model input that is input to the input layer in an iteration of the iterative process may represent a sub-volume of the image data.

The sub-volume may be dependent on the position and/or orientation and/or steering prediction determined in one or several preceding iterations of the iterative process.

An orientation of the sub-volume may be dependent on the preceding steering prediction.

A center of the sub-volume may be shifted relative to the previously determined point by an offset.

The offset may be dependent on the steering prediction determined in the preceding iteration.

The method may further comprise a multivariate interpolation of the pixel or voxel values of the image data to determine the AI model input.

The multivariate interpolation may comprise weighted averaging of the pixel or voxel values to generate the AI model input as interpolated pixel or voxel values on a grid that is tilted and/or offset relative to a pixel or voxel grid of the image data.

The multivariate interpolation may comprise a multilinear projection.

The method may further comprise performing an initialization of the iterative procedure.

The initialization may be dependent on whether an initial direction is available.

When the initial direction is not available, the method may comprise determining the initial direction for use in an initial iteration of the iterative process.

Determining the initial direction may comprise determining uncertainty estimates of predictions for various directions.

Determining the initial direction may comprise the direction and steering prediction from among the various directions for which the uncertainty estimate in the corresponding prediction is minimum.

The uncertainty estimates may be determined using Monte-Carlo dropout.

The AI model may comprise a convolutional neural network, CNN, or a recurrent neural network, RNN.

The AI model may comprise the input layer, a plurality of convolutional layers, a dropout layer or several dropout layers, and the output layer.

The AI model may comprise one or several of pooling layers, attention modules, self-attention modules, atrous convolutions, normalization layers, batch normalization layers, transformers, recurrent layers.

One, several, or all of the following may apply: the output layer is a linear layer; the plurality of convolutional layers comprises one or several strided convolutional layers; the AI model is or comprises a fully convolutional network; the AI model comprises one or several nonlinearities; the AI model comprises exponential linear units (ELU); the AI model comprises rectified linear units (ReLU); the AI model comprises Scaled Exponential Linear units (SELU); the AI model comprises Gaussian Error Linear Unit (GELU).

The object may be an object extending along a time axis of the image data.

The object may be an object in a video.

The object may be an object that is to be traced through a series of image frames.

The object may be a biological, in particular physiological, object.

The object may be a human or animal neurite.

The object may be a human or animal blood vessel.

The object may be an asset of an infrastructure, in particular utility, system.

The object may be a tube of a fresh water or sewage water system.

The object may be a tubing of an industrial plant.

The object may have a surface that extends around a center axis of the object in a locally cylindrical manner.

The method may further comprise training the AI model.

The AI model may be trained using supervised learning.

Training the AI model may comprise gradient-based updates of AI model parameters.

Training the AI model may comprise training the AI model to comply with a flight policy that causes the iterative process to converge to the object when starting from or reaching a location in the image data that is off the object.

A convergence distance of the flight policy may determine how rapidly the iterative process converges back to the object.

The convergence distance may be a dynamic parameter that may be varied during AI model training.

The convergence distance may be set as a function of a distance from a boundary represented by the image data during AI model training.

The convergence distance may be a monotonously increasing function of the distance from the boundary.

An image processing method may comprise receiving and/or acquiring image data and performing the object tracing method.

The image processing method may comprise outputting one or several of: a visual representation of the series of points or of the center line; a machine-readable representation of the series of points or of the center line; a control signal that is dependent on the series of points located along the center line; a machine learning, ML, model that has an architecture that depends on the series of points located along the center line.

The image data may comprise a time series of time-sequential image frames.

Tracing the object may comprise tracing a physical object along a time axis in a spatiotemporal coordinate system.

A control method comprises the object tracing method or image processing method, and further comprises controlling at least one controllable asset based on a result of the object tracing method.

There is also disclosed a processing system that comprises an interface to receive image data and at least one circuit (e.g., a set of processors or other integrated circuits) operative to perform the object tracing method or image processing method.

There is also disclosed a system that comprises at least one controllable asset (e.g., a machine or actuator) and the processing system, wherein the controllable asset is responsive to a control command or control signal generated by the processing system.

Various effects and advantages are attained by the methods and systems according to embodiments of the invention. The methods and systems allow elongate objects to be traced with a low error rate. The methods and systems facilitate the automatic analysis of images, e.g., of images of neural tissue, for purposes such as connectomics.

The methods and systems allow neurites to be traced with an accuracy that is comparable to or outperforms conventional automatic or semi-automatic techniques and that approaches that of human annotators. The methods and systems are applicable not only to dendrite tracing but can also trace, e.g., axons and spine necks, which are typically harder to trace than dendrites.

According to additional aspects of the invention, there are also disclosed image processing methods and image processing systems operative to perform a local realignment, which may comprise the following steps executed to perform the local realignment of slices of image data along a slicing direction: determining slice-to-slice shift vectors for a plurality of slices along the slicing direction; identifying valid slice-to-slice shift vectors between slices; using the valid slice-to-slice shift vectors to generate a locally realigned subvolume.

The locally realigned subvolume may be fed to the AI model input, e.g., to an input layer of a CNN or RNN or to a filter or other preprocessing that preprocesses the realigned subvolume prior to it being processed by the CNN or RNN.

Determining the slice-to-slice shift vectors may comprise determining auto- and cross-correlation of slices.

Determining the slice-to-slice shift vectors and/or their validity may comprise determining a mean and a standard deviation of correlation-weighted shift vectors based on a first threshold criterion.

Identifying valid slice-to-slice shift vectors may comprise verifying that the determined auto-correlations fulfill a first criterion (such as having a peak at a desired shift, e.g., at zero shift).

Identifying valid slice-to-slice shift vectors may comprise verifying that the determined auto-correlation peak has a standard deviation that fulfills a second criterion (such as having a standard deviation that fulfils a second threshold criterion, e.g., being less than or equal to a certain number of pixels).

Identifying valid slice-to-slice shift vectors may comprise verifying that a combination of two successive auto- and/or cross-correlations peaks fulfils a third criterion (e.g., fulfils a third threshold criterion, where the third threshold criterion may optionally be dependent on surrounding auto- and/or cross-correlations).

Identifying valid slice-to-slice shift vectors may comprise verifying that the determined cross-correlation fulfills a first cross-correlation criterion that depends on auto-correlations for the two involved slices (such as the mentioned auto-correlation criteria being valid for both slices).

Identifying valid slice-to-slice shift vectors may comprise verifying that the determined cross-correlation normalized with the expected cross-correlation value for fully correlated intensities fulfills a second cross-correlation criterion (such as a peak of the cross-correlation normalized with the expected cross-correlation value for fully correlated intensities fulfilling yet another threshold criterion, such as being equal to or greater than a fourth threshold).

Identifying valid slice-to-slice shift vectors may comprise verifying that a standard deviation of the shift vector fulfills a third cross-correlation criterion (such as the standard deviation being equal to or less than a fifth threshold).

Using the valid shift vectors may comprise identifying one or several slices to be realigned even though the slice-to-slice shift vector is not considered valid, and computing the shift vector in this case from valid shift vectors relating to a neighborhood of these one or several slices.

Using the valid shift vectors may comprise generating a locally realigned and trilinearly interpolated subvolume.

Generating the locally realigned and trilinearly interpolated subvolume may comprise performing floor or ceil operations.

Generating the locally realigned and trilinearly interpolated subvolume may comprise performing a trilinear interpolation as a sequence of linear interpolations along cardinal axes.

The sequence of linear interpolations may comprise four linear interpolations along a first dimension, two linear interpolations along a second dimension, and one linear interpolation along a third dimension.

The locally realigned subvolume may be processed to trace an elongate object, such as a blood vessel or neurite, in 3D.

The local realignment techniques may be used in association with (e.g., as a part of) the methods of tracing an elongate object, without being limited thereto. These local realignment techniques address for referencing slices to a common reference frame in a particularly efficient manner.

### BRIEF DESCRIPTION OF THE DRAWINGS

Embodiments will be described in detail with reference to the drawings in which identical or similar components are designated by identical or similar reference signs.

FIG. 1 is a block diagram representation of a system according to an embodiment.

FIG. 2 shows an elongate object in 3D image data.

FIG. 3 shows an elongate object in 3D image data.

FIG. 4 is a view of a series of points for explaining operation of systems and methods according to embodiments.

FIG. 5 is a flow chart of a method according to an embodiment.

FIG. 6 is a view of sub-volumes of an imaged tissue volume for explaining operation of systems and methods according to embodiments.

FIG. 7 is a view of voxel grids.

FIG. 8 is a view illustrating determination of an artificial intelligence (AI) model input.

FIG. 9 shows an AI model of systems and methods according to embodiments.

FIG. 10 shows an AI model of systems and methods according to embodiments.

FIG. 11 is a flow chart of a method according to an embodiment.

FIG. 12 is a flow chart of a method according to an embodiment.

FIG. 13 shows an AI model of systems and methods according to embodiments and illustrates operation thereof.

FIG. 14 shows a further AI model of systems and methods according to embodiments and illustrates operation thereof.

FIG. 15 is a flow chart of a method according to an embodiment.

FIG. 16 is a view illustrating a flight policy that may be employed during AI model training.

FIG. 17 is a flow chart of a method according to an embodiment.

FIG. 18 is a flow chart of a method according to an embodiment.

FIG. 19 illustrates operation of systems and methods according to embodiments.

FIG. 20 illustrates a local coordinate frame that may be employed by methods and systems according to embodiments.

FIG. 21 shows an error rate of methods and systems according to embodiments as a function of training iterations.

FIG. 22 is a flow chart of a method according to an embodiment.

FIG. 23 is a diagram showing the applications to which embodiments of the invention lend themselves due to their low reconstruction error rate and large distance between reconstruction errors.

FIG. 24 is a diagram showing the improvements attained by embodiments as compared to conventional techniques.

FIG. 25 is a diagram showing the improvements attained by embodiments as compared to conventional techniques.

FIG. 26 is a further diagram showing the improvements attained by embodiments as compared to conventional techniques.

FIG. 27 is a flow chart of a method according to an embodiment.

FIG. 28 is a block diagram representation of a system according to an embodiment.

### DETAILED DESCRIPTION OF EMBODIMENTS

Embodiments will be described in detail with reference to the drawings. In the drawings, identical or similar components are designated by identical or similar reference numerals.

While embodiments will be described in detail with reference to specific physiological objects, such as neurites, axons, and spine necks, the techniques disclosed herein are not limited thereto. Methods and systems operative as disclosed herein may be used for processing image data for tracing a variety of physiological objects, such as blood vessel or other elongate lumen walls. Methods and systems operative as disclosed herein may also be used to generate annotated image data from raw image data.

While embodiments will be described in detail with reference to specific imaging techniques or specific image data, such as three-dimensional electron microscopy (3D EM) imaging and 3D EM images, the methods and systems may also be used to process images acquired with other imaging techniques, such as light microscopy, magnetic resonance imaging, X-ray holographic nano-tomography, etc.

### Elongate Object Tracing

The systems and methods disclosed herein are operative to trace an elongate object (such as a neurite, without being limited thereto) in image data. The image data may be 3D image data or may be image data in a spatio-temporal coordinate system that has the time axis as one of its axes. The image data may be biological or medical image data. The image data may include a series of frames that may be taken time-sequentially. The techniques disclosed herein are applicable to other image data as well.

The systems and methods can determine a series of points that are predicted to lie on a center line of the elongate object, to thereby trace the object. The systems and methods may be operative to process voxel or pixel values of image data, without requiring prior object recognition in the image data.

The systems and methods disclosed herein may perform object tracing by (a) selecting a sub-volume of the image data, (b) determining information on a direction along which the elongate object extends in the sub-volume, and (c) processing the direction to determine a further point located along the center axis. These steps may be repeated iteratively.

The sub-volume that is processed in an iteration may have an orientation that depends on the direction of extension of the object determined in a preceding iteration. Thus, the direction steers the analysis process, akin to autonomous driving or flight techniques. The direction along which the center axis of the elongate object extends is therefore also referred to as “flight direction” herein. This direction determines the path along which the techniques disclosed herein operate.

Changes in the flight direction are determined at least by a steering prediction. The steering prediction may define or otherwise relate to a derivative (e.g., a first or second or higher order derivative) of the flight direction. As used herein, the term “steering prediction” refers to a quantity that depends on how the elongate object extends from a previously determined point along the elongate object and/or that determines how (in particular with which sub-volume orientation and/or location) the iterative technique disclosed herein is to proceed.

An orientation of a sub-volume that is analyzed next may be set in dependence on the flight direction and/or steering prediction.

The determination of the steering prediction may use an AI model. The AI model may have an input layer operative to receive voxel values determined from the image data. The AI model input may be taken from the image data or, more generally, may be obtained by interpolating adjacent voxels of the image data to accommodate arbitrary positions and orientations of the sub-volume. The AI model may have an output layer operative to output the steering prediction. For enhanced accuracy and stability, the AI model may be an AI model that has been trained not only on positions located along the center line of elongate objects in training image data, but also on positions located off the center line of elongate objects in the training image data. Convergence back to the center line can thereby be ensured also during inference.

The determination of the new point along the center line may be performed using an integration of the steering prediction. The integration may comprise several subsequent integrations, e.g., when the steering prediction relates to a higher order derivative of the center line direction of the elongate object.

The systems and methods are independent of the affinity prediction of individual voxels and do not require an explicit volume segmentation of the image data. Instead, the systems and methods are operative to directly solve the problem of following the center line of elongate objects such as neuronal wires. The systems and methods facilitate large-scale connectomic reconstruction. Since they are also computationally efficient, resource consumption for acquiring connectomes from animals' and human brains, in health and disease, for screening of circuit disorders and bioptic analysis of pathological tissue in the clinical setting are facilitated.

The systems and methods provide an approach for automated axon reconstruction in 3D EM data by training an AI model on mimicking axon flight mode tracing. The systems and methods may employ a convolutional neural network (CNN). Details of CNNs are described in, e.g., Y. LeCun et al. 1989. “Backpropagation Applied to Handwritten Zip Code Recognition.” Neural Computation 1 (4): 541-51.

The systems and methods provide various differences and effects as compared to the CNN-based techniques of M. Januszewski et al. 2018. “High-Precision Automated Reconstruction of Neurons with Flood-Filling Networks.” Nature Methods. https://doi.org/10.1038/s41592-018-0049-4.

The systems and methods determine the (approximate) center line of elongate objects such as neurites and through iterations skeleton-reconstructs the center line. The sub-volume selection (such as sub-volume orientation) and problem formulation ensure the analysis to be aligned to the elongate axis. Only the deviation of the center line from going straight (along the sub-volume orientation) must be predicted in order to stay aligned.

I.e., the systems and methods disclosed herein analyze a sub-volume, in each of several iterations of an iterative process, that is oriented in a manner that takes into account the local direction of the elongate object.

If the center line is straight, the steering prediction is zero. If the center line deviates from being straight, the steering prediction may capture this deviation by means of, e.g., a parabola approximation to the neurite at the current location.

By comparison, the sub-volumes of the Flood-Filling Networks (FFN) of M. Januszewski et al. 2018. “High-Precision Automated Reconstruction of Neurons with Flood-Filling Networks.” Nature Methods. https://doi.org/10.1038/s41592-018-0049-4 are oriented along the cardinal axes of the image dataset and therefore do not capture the main axis/center line orientation of the neurite. These conventional techniques do not provide a mechanism by which a main axis or center line would be captured or used for the reconstruction. Instead FFN predicts the affinity of each voxel, which adds to the computational complexity and computational costs. Thus, FFN is computationally more expensive than the techniques disclosed herein. FFN is restricted to integer coordinates, while the techniques disclosed herein afford multivariate interpolation of image data and a continuous problem formulation in terms of a spatially varying coordinate frame, which is more readily applicable to continuous coordinates for input and/or output.

For the techniques disclosed herein, the AI model is trained on the task of predicting the local continuation of the center line from, e.g., a neurite-centered and -aligned 3D-EM sub-volume. This allows for direct optimization of the error rate, a metric on which conventional automated methods have made little progress and a metric, which exceeds voxel-based metrics in relevance for the extraction of connectomes from 3D-EM volumes that are much larger than typical neurite diameters.

During inference, the systems and methods are operative to integrate the steering prediction to a new position and direction, such that recurrent application yields a trace of visited positions similar to the skeletons generated by human annotations.

Directionality and continuity of elongate objects (such as neurites) may be incorporated directly into the problem formulation.

Tests show that the techniques disclosed herein yield low error rates even for lower resolution data with significantly reduced computational cost compared to previous methods. In addition, the methods and systems obviate any need for a segmentation and can operate on the 3D image data without prior segmentation.

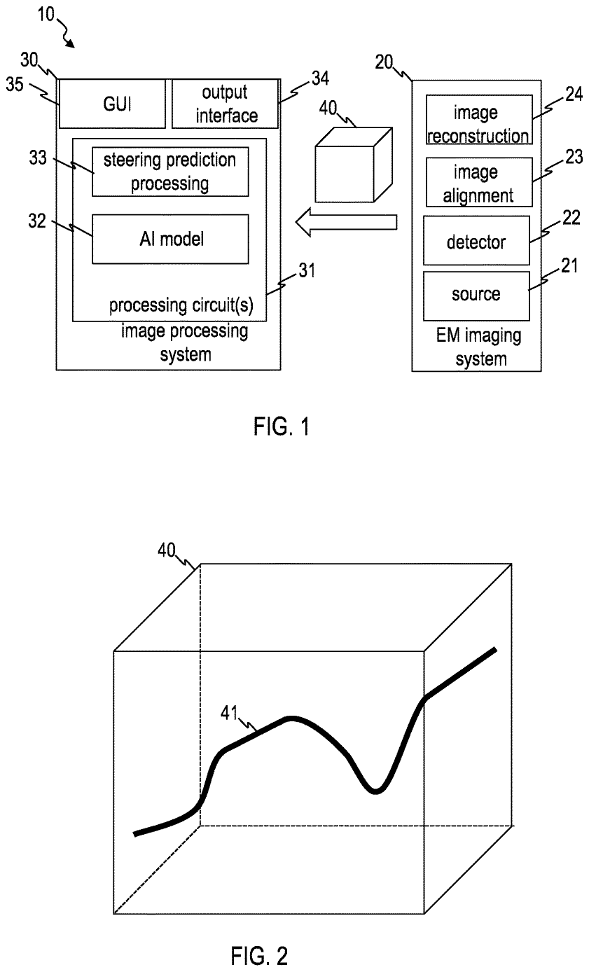

FIG. 1 is a block diagram of a system 10. The system 10 is operative to trace an elongate object (such as a neurite) in 3D image data (such as 3D-EM image data).

The system comprises an imaging system 20. The imaging system 20 may be a 3D EM imaging system. The imaging system 20 may comprise a source 21. The source 21 may comprise an electron source that may be operative to output a beam of electrons, for example. The imaging system 20 may comprise a detector 22. The detector 22 may be operative to detect electrons that have been scattered, reflected, or refracted off physiological objects within a sample (such as a biopsy sample). The imaging system 20 may comprise an image alignment component 23 for image alignment prior to reconstruction.

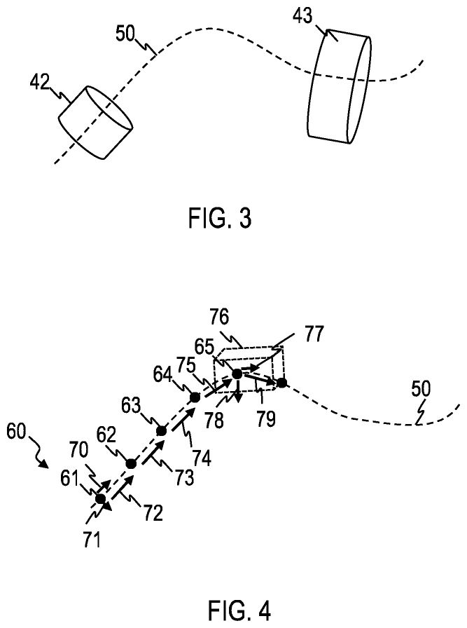

The imaging system 20 may be operative to perform a local realignment. The local realignment may be performed by image alignment component 23 or the imaging system 20 may be operative to execute a second, local image realignment component in addition to the image alignment component 23. The imaging system 20 (and in particular its local image realignment component) may be operative to perform a local re-alignment and de-alignment for mapping to a global image coordinate system. For illustration, the imaging system 20 may be operative to perform the method of P. H. Li et al., “Automated Reconstruction of a Serial-Section EM Drosophila Brain with Flood-Filling Networks and Local Realignment.” Preprint. https://doi.org/10.1101/605634, in particular the techniques described in association with FIG. 3 of this preprint.

The local realignment may be performed using techniques as specifically described in greater detail hereinbelow.

The imaging system 20 may comprise an image reconstruction circuit 24. The image reconstruction circuit 24 may be operative to compute voxels representing 3D image data of the sample. The 3D image data 40 may be provided to image processing system 30. Instead of or in addition to 3D image data, other forms of a volumetric tissue representation may be used, such as a series of two-dimensional (2D) images or projections. An example for such a representation is X-ray holographic nano-tomography (which may be performed using, e.g., the techniques of A. T. Kuan et al., “Dense neuronal reconstruction through X-ray holographic nano-tomography”, Nature Neuroscience volume 23, pages 1637-1643 (2020).

The image processing system 30 comprises one or several processing circuit(s) 31. The processing circuit(s) 31 may include one or several circuits, including integrated circuits such as field programmable gate arrays (FPGAs), application specific integrated circuits (ASICs), controller(s), processor(s), superconducting circuit(s), quantum bit (qubit) processing circuits, without being limited thereto.

The processing circuit(s) 31 may be operative to perform an elongate object tracing to follow an elongate object (such as a neurite, without being limited thereto) through the volumetric representation 40 of tissue. Starting from a position on a center line of the elongate object, an AI model 32 may respectively be fed with AI model input that depends on (and may represent) a sub-volume of the image data 40. The AI model 32 is trained to output, responsive to this image data, a direction along which the elongate object extends from the already known point. This direction determines the direction in which the tracing is continued and is therefore also referred to as steering prediction. Based on the steering prediction, a next point (adjacent to the already known point) along the center line is computed. This may be done by integration that may be performed by steering prediction processing 33. The determination of the steering prediction by the AI model and the determination of a new point along the center line by processing of the steering prediction may be repeated to thereby generate a series of points located on the center line.

No segmentation of the image data 40 is required for the system or method to operate in the manner disclosed herein.

Determining the points located along the center line in 3D respectively comprises determining the location of the points within the 3D volume represented by the image data 40. Determining the points includes determining information that allows all three coordinates of the points to be specified in the volume represented by the image data 40. Determining the points may include determining three cartesian coordinates of the points, it being understood that many equivalent formulations may be used to specify the position of a point in 3D.

A result of the image processing may be output by the image processing system 30. The image processing system 30 may comprise a graphical user interface (GUI) to output a graphical representation of the series of points and/or the center line of the elongate object. The GUI may be operative to output a visualization of the center line of the elongate object. Alternatively or additionally, the image processing system 30 may comprise an output interface 34. The output interface 34 may be operative to output machine-readable data that depend on a result of the elongate object tracing. The machine-readable data may comprise control signals that depend on a result of the elongate object tracing. The machine-readable data may comprise a control logic that depends on a result of the elongate object tracing, for example when using the results of neurite tracing for neuroscience-inspired machine learning (ML) model design or training.

The image processing system 30 may also be operative to control the GUI 30 to allow a user to enter a starting point for the object tracing. The image processing system 30 may be operative to control the GUI 30 to allow a user to optionally enter a starting direction for the object tracing. Knowledge of the initial direction is optional, as explained in more detail herein. An initial direction may be determined automatically, using, e.g., uncertainty estimates obtained from Monte-Carlo dropout.

While elongate object tracing is further illustrated in association with tracing of one elongate object in the following, the systems and methods are operative to trace plural elongate objects. The techniques are also applicable to plural elongate objects and/or plural elongate parts of objects and/or plural parts of elongate objects. This may be done in a parallel or sequential manner. For illustration, plural instances of the image processing techniques disclosed herein may be instantiated to concurrently trace plural elongate objects in parallel. Alternatively or additionally, the systems and methods may loop through the techniques disclosed herein several times for different elongate objects. This may be done until the 3D trace has been determined for each desired elongate object. For illustration, the 3D traces of plural (e.g., in excess of 100 or in excess of 1000) neurites, axons, and/or neural spine necks may be traced. The methods disclosed herein lend themselves to use in connectomics, without being limited thereto.

FIG. 2 shows a volume 40 represented by image data and an elongate object 41 extending therein. The systems and methods disclosed herein are operative to trace a center line of the elongate object 41. The systems and methods may use steering-prediction dependent selection of sub-volumes, AI model processing of the image data in the sub-volume for updating the steering prediction, and integration of the steering prediction to follow a center line of the elongate object 41. The systems and methods may employ these techniques for neurite tracing, without being limited thereto.

FIG. 3 shows a center line 50 of an elongate object. The techniques disclosed herein are not only applicable to neurites but are more broadly applicable to other objects such as blood vessels, lumina of the respiratory tract, etc. The techniques are also applicable to traces in a spatio-temporal coordinate system (with the physical object tracing an elongate path along the time axis not necessarily being physically elongate). The techniques are also applicable to plural elongate objects and/or plural elongate parts of objects and/or plural parts of elongate objects.

The methods and systems disclosed herein may be used to trace an object (e.g., a center line 50 of an elongate object) that has a locally cylindrical outer shape. The term “locally cylindrical” is used to refer to a geometry in which outer walls of the elongate object can be locally approximated by cylinder segments 42, 43. The orientation of the various cylinder segments 42, 43 may be tilted relative to each other in 3D space. The diameter of the cylinder segments 42, 43 may vary along the center line 50 (as is also typically the case for neurites).

FIG. 4 illustrates operation of the methods and systems disclosed herein. Processing starts with image data and an initial point 61 located on an elongate object that is to be traced. The initial point 61 may be set by a user, also to identify which object is to be traced. Alternatively, automatic object recognition may be employed to identify a starting point 61 on an object. An initial flight direction 70 that specifies the direction of the elongate object at the starting point 61 may be received by a user input or may be estimated automatically by the system, using, e.g., prediction uncertainties that specify uncertainties of prediction estimates for different directions (as obtainable from Monte-Carlo dropout, without being limited thereto).

The systems and methods are operative to
identify a first sub-volume that is located around (but typically has a first center somewhat offset from) the starting point 61,
determine a first steering prediction 71 by processing image information from the first sub-volume, and
processing at least the first steering prediction 71 (e.g., using integration) to determine
   a position offset 72 giving the first point 62 subsequent to the starting point 61,
   a new sub-volume orientation (not shown) for the subsequent iteration.

These steps may utilize the starting point 61 and starting direction 70 as input.

The process may then be repeated in an iterative manner. For illustration, the systems and methods may be operative to then
identify a second sub-volume that is located around (but typically has a second center somewhat offset from) the first point 62,
determine a second steering prediction by processing image information from the second sub-volume, and
determine a second point 63 subsequent to the first point 62 by processing at least the second steering prediction (e.g., using integration) and determining a position offset 73 and a new sub-volume orientation (not shown).

The process may be repeated in a recurrent manner to determine a series 60 of points located on the center line 50 by recurrent inference. In this way, additional steering predictions, yielding additional position offsets 74, 75, 79 and additional points 64, 65 of the series 60 may be determined.

In an iteration of the iterative process, a sub-volume 76 of the volume represented by the image data 40 is determined. The sub-volume 76 contains the current point 65 determined in the preceding iteration. The sub-volume 76 has an orientation that depends on the preceding steering prediction determined in the preceding iteration. Image data for the sub-volume 76 is processed by the AI model to determine a steering prediction 78 for the current iteration of the iterative process. The next point and orientation that follow the current point 65 are determined using the steering prediction 78 for the current iteration, e.g., by integration (such as by adding a vector proportional to the momentary flight direction 77 to the current point 65 to determine the next point and by adding a vector proportional to the steering prediction to the momentary flight direction/sub-volume orientation 77 to determine the next flight direction/sub-volume orientation, or alternatively by using more complex extrapolation techniques to extrapolation from the series of previously determined steering predictions). As previously discussed, analysis of the image data contained in the sub-volume 76 provides the steering prediction 78, which is integrated to determine a next position offset 79 to continue tracing the elongate object.

The systems and methods disclosed herein may thereby perform processing of sub-volumes of the volume 40 to infer the local direction of the center line 50, which corresponds to the steering prediction. Each sub-volume may be centered, in the two directions transverse to the momentary flight direction/sub-volume orientation, around the current point determined in the preceding iteration. Each sub-volume may have a center that is offset from the preceding point determined in the preceding iteration, the offset being in the direction parallel to the momentary flight direction. For illustration, the sub-volume 76 may be positioned such that the point 65 determined in the preceding iteration is closer to the end face of the sub-volume 76 that is located from the point 65 in the direction opposite the momentary flight direction 77, than to the other end face of the sub-volume 76 that is located from the point 65 in the direction along the momentary flight direction 77.

The methods and systems disclosed herein thus employ an approach in which the tracing process is automatically steered along the center line, with a field of view (i.e., sub-volume) that is being analyzed at each step being directed along the estimated tangential of the center line. Continuity of the elongate object is automatically built in the process. The process is akin to tracing a desired path in autonomous driving (but in 3D).

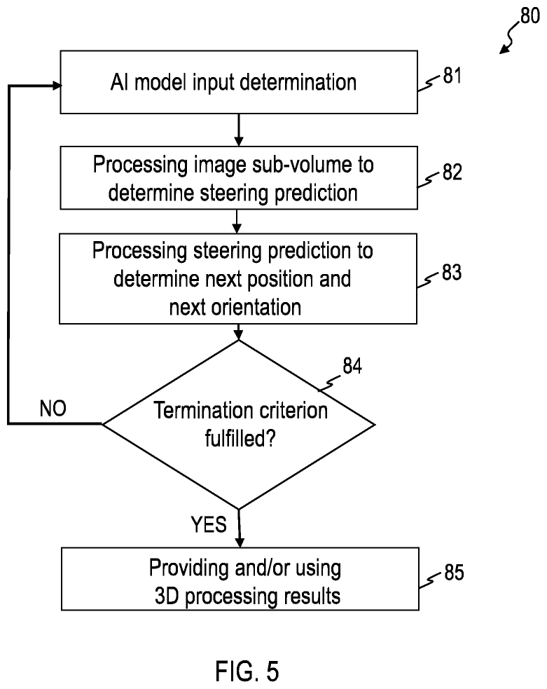

FIG. 5 is a flow chart of a method 80. The method 80 may be performed automatically by the image processing system. The method 80 determines a series of points 60 along the center line 50.

At step 81, AI model input is determined from the image data and the current point. In a first iteration, the current point is the starting point 61 that may be set by a user input. In each subsequent iteration, the current point may be the point 62-65 on the center line determined in the preceding iteration.

The determination of the AI model input at step 81 may determine the AI model input as image data from a sub-volume 76 of volume 40. The sub-volume 76 may contain a current point that may be a result of a preceding iteration. The sub-volume 76 may have an orientation that depends on a preceding steering prediction and sub-volume orientation. The sub-volume 76 may (and generally is) tilted relative to edges of the volume 40. In a first iteration, the sub-volume orientation may be determined based on a user input, based on calculated prediction uncertainties over different orientations, as a random orientation, or otherwise. In each subsequent iteration, the current position and sub-volume orientation is the one determined by the AI model in the preceding iteration of the method 80.

Determination of the AI model input at step 81 may comprise averaging (e.g., weighted averaging) or other interpolation techniques performed on voxel or pixel values of the image data 40, explained in more detail herein.

At step 82, the sub-volume of the image is processed. This may comprise processing the AI model input with the AI model. The output layer of the AI model may output an updated steering prediction.

At step 83, at least the steering prediction obtained at step 82 is processed to determine a next point along the center line 50 and a next orientation for the sub-volume. This may comprise vector addition or integration of the preceding (and optionally, several previously determined) steering predictions, positions and orientations.

At step 84, it is determined whether a termination criterion is fulfilled. Termination criteria may be any one or any combination of the following without being limited thereto: intersection of the sub-volume and or next position to be analyzed with a boundary of the image data 40 or a user-defined boundary; threshold criterion for number of iterations or accumulated path length; no identifiable continuation of the elongate object in the sub-volume; intersection of the position or accumulation of path length within an object defined by other means (e.g., intersection of the neurite that is being traced with a previously traced neurite). If the termination criterion is not fulfilled, the method may return to step 81. Otherwise, a result of the elongate object tracing may be output via a GUI or data interface or may be otherwise used at step 85.

The technique of FIG. 5 may be used as part of a more complex process or method. can also be embedded in some higher order logic. For illustration, a method is disclosed that automatically detects missing continuation of an automated reconstruction, starts the technique of FIG. 5 responsive to the detection of this missing continuation at that location and run the technique of FIG. 5 until it finds another automatically reconstructed component. From that point, the technique disclosed herein (and as explained with reference to FIG. 5) may then be run in a backward direction to reconfirm that it ends up at the same start location.

Thus, the technique of FIG. 5 can be used as a building block for more complex reconstruction methods.

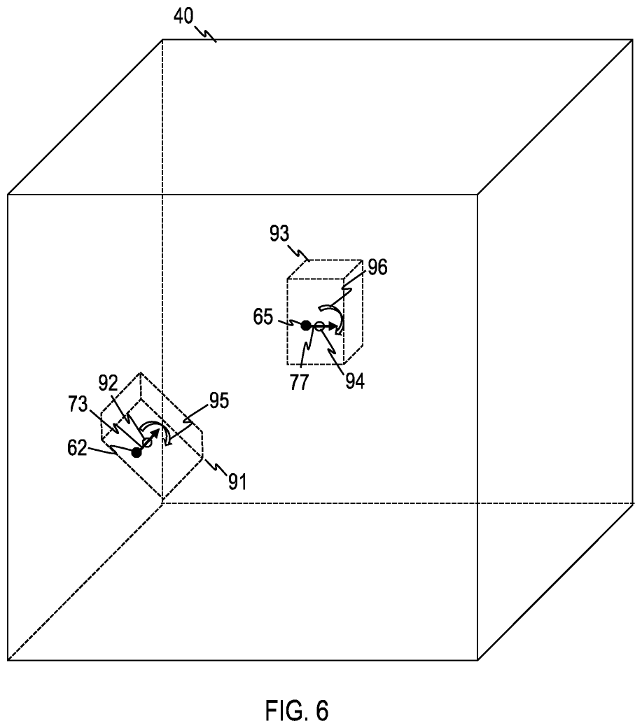

FIG. 6 illustrates the location-dependent selection of a sub-volume for analysis in the systems and methods disclosed herein. The point 62 along the center line 50 and momentary flight direction 71 determined in an iteration of the recurrent inference process are used to determine which portion of the volume 40 is to be processed by the AI model next to continue tracing of the elongate object. A sub-volume 91 may have a cuboid shape, without being limited thereto. Four of the edges of the sub-volume 91 may be parallel to the momentary flight direction 71. The four edges parallel to the steering prediction 71 may be chosen constantly or dependent on previous AI model predictions shorter, longer or same length as the other edges of the sub-volume 91. The sub-volume 91 may have a size that depends on the object that is to be traced.

The point 62 may be contained within the sub-volume 91. The sub-volume 91 may be positioned such that its center 92 is offset from the point 62 by an offset along the momentary flight direction 73. I.e., the sub-volume may be positioned such that it extends a greater distance along the momentary flight direction 73 than opposite the momentary flight direction, as seen from the point 62.

The orientation of the sub-volume is variable, depending on the previous flight direction and steering prediction determined in the preceding iteration.

For illustration, point 65 along the center line 50 and momentary flight direction 77 determined in an iteration of the recurrent inference process are used to determine which portion of the volume 40 is to be processed by the AI model next to continue tracing of the elongate object. A sub-volume 93 may have a cuboid shape, without being limited thereto. Four of the edges of the sub-volume 93 may be parallel to the momentary flight direction 77. The four edges parallel to the momentary flight direction 77 may be shorter, longer or same length as the other edges of the sub-volume 93 and can either be chosen as a constant or dependent on the AI model predictions from the previous iteration.

The point 65 may be contained within the sub-volume 93. The sub-volume 93 may be positioned such that its center 94 is offset from the point 65 by an offset along the momentary flight direction 77. I.e., the sub-volume may be positioned such that it extends a greater distance along the momentary flight direction 77 than opposite the steering prediction, as seen from the point 65.

The orientation of the edges of the sub-volumes 91, 93 that are not parallel to the momentary flight directions 73, 77 are not germane. A random rotation 95, 96 around the momentary flight direction 73, 77 may be introduced during training and/or during inference when selecting the sub-volumes 91, 93. This further enhances robustness of the techniques disclosed herein.

The random rotation 95, 96 may be replaced or combined with methods that make the AI model (approximately) equivariant to this rotation. This has the effect that feeding a rotated input yields an accordingly rotated steering prediction. This may further increase robustness of the techniques disclosed herein.

The edges of the sub-volumes 91, 93 that are parallel to the steering direction may be chosen constantly or dependent on previous AI model predictions shorter, longer or same length as the edges of the sub-volumes 91, 93 that are not parallel to the steering direction. This further enhances robustness of the techniques disclosed herein.

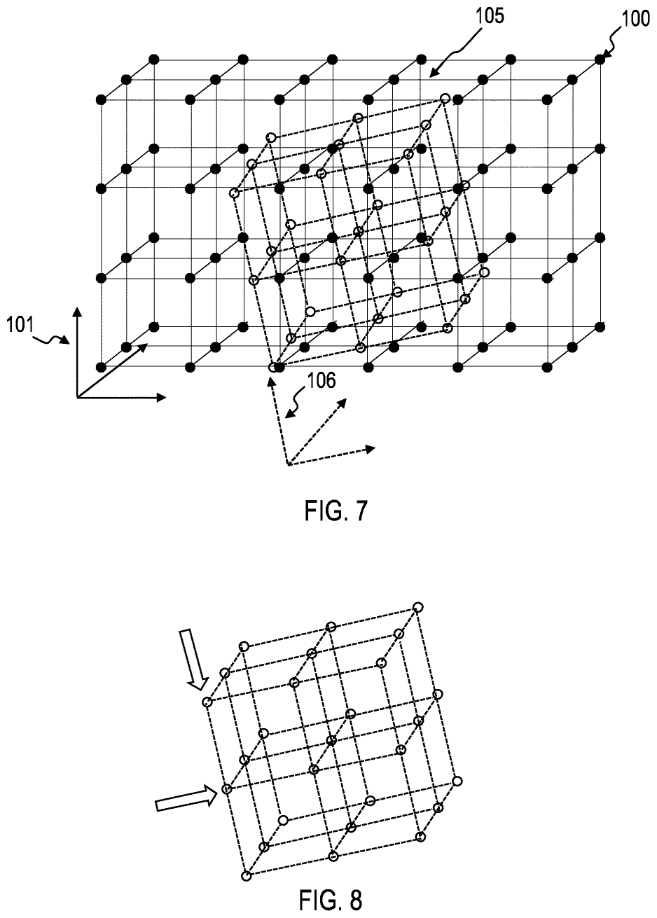

The dependence of sub-volumes 91, 93 on the preceding steering prediction, preceding flight direction and preceding points has the effect that voxels of the sub-volumes 91, 93 will typically not coincide with voxels of the original image data 40. Interpolation may be used to compute the voxel values within the sub-volumes 91, 93 for arbitrary orientation and position of the sub-volumes 91, 93, as illustrated in FIGS. 7 and 8.

FIGS. 7 and 8 show voxels of the image data 40 (e.g., centers of the voxels) with solid circles. Voxels of a sub-volume 91, 93 are shown by open circles and can in general be anisotropic in terms of physical length scales. The voxels of the image data 40 define a grid 100. The voxels of the sub-volume define a second grid 105, which can be anisotropic. Second coordinate axes 106 of the second grid 105 may be rotated in 3D and/or shifted in 3D relative to coordinate axes 105 of the grid 100 on which the voxels of the original image data 40 are defined.

Interpolation techniques may be used to determine a voxel value (e.g., as a grayscale or color value with various color channels) on a location on the second grid 105, which is then one of the AI model inputs.

Projection may be used to determine the voxel values on the second grid 105. For illustration, as shown in FIG. 8, projection (such as a trilinear projection) may be used to project the image data onto a plane of the grid 105.

Other techniques may be used. For illustration, a point on the second grid 105 (i.e., a voxel center of the sub-volume 91, 93) may be located within a cube defined by several (e.g., eight) voxel centers 103 of the original image data 40. Weighted averaging may be performed to compute the voxel value for the point 106 of the sub-volume 91, 93 from the voxel values of the original image data. For illustration, the voxel value for the point 106 may be computed as a weighted average of the voxel values of the eight voxel centers 103 located around the point on the second grid 105. Other techniques may be used to interpolate the image data to the grid 105 defined for the sub-volume, to thereby determine the AI model inputs, such as trilinear projections (as described above).

As already mentioned, and as discussed in more detail hereinbelow, local realignment may be applied. In FIG. 7, local realignment may be applied with respect to (potentially non-integer) x and/or y offsets (which may be obtained from cross-correlation) of 2D image planes (shown by planes of black dots in FIG. 7). Hence, a trilinear interpolation (for generating voxel values at the white dot positions in FIG. 7, when voxels at black dot positions are subject to local realignment) can be computed using two bilinear interpolations per white dot position separately determined on floored and ceiled z coordinates with potentially different x/y offsets applied to the black dot positions and then combining those two intermediate values from the bilinear interpolations by another linear interpolation along the z axis.

With regard to AI model configuration, an AI model that includes convolutional layers has been determined to provide good performance. For illustration, the AI model may be or may comprise a CNN.

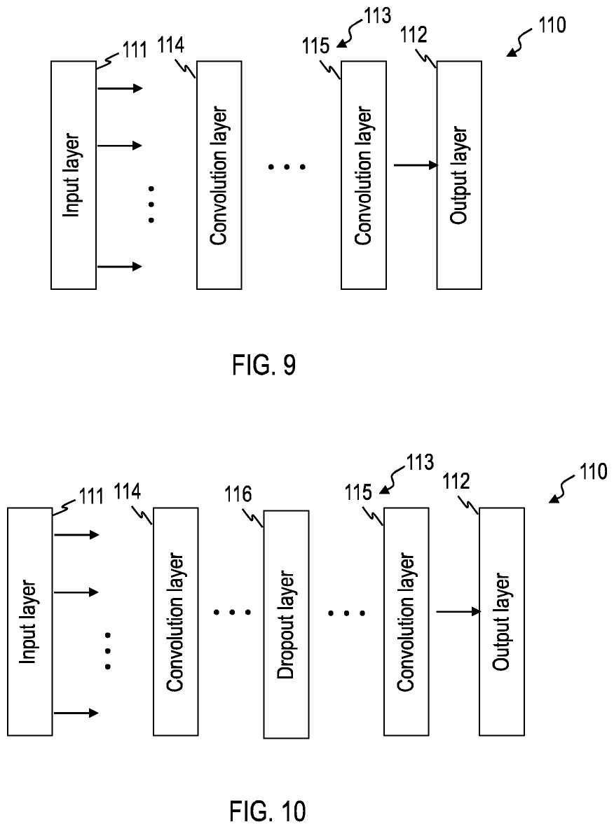

FIGS. 9 and 10 show AI models that may be used in systems and methods disclosed herein.

The AI model 110 comprises an input layer 111 operative to receive an AI model input that is based on pixel or voxel values of the image data. The AI model input may comprise or may be image data within the sub-volume 91, 93, as determined from the voxel values of the image data 40. The voxel values may be converted from integers to floating point representation and normalized before being processed by the AI model. The normalization can be applied to limit the AI model input values to a certain range and/or to change statistical properties of the AI model input values, such as to achieve a mean input value of zero and a standard deviation over input values of one.

The AI model 110 comprises an output layer 112 operative to provide an AI model output that is or indicates the steering prediction 71-75.

The AI model 110 comprises hidden layers 113. The hidden layers 113 may comprise several convolutional layers 114, 115. The hidden layers 113 may comprise strided convolutional layers and/or fully connected layers. Strides may be different for different convolutional layers.

The hidden layers 113 may comprise several convolutional layers 114, a dropout and/or reshaping layer 116, and one or several fully connected layers 115.

The hidden layers 113 may comprise a linear layer as last layer.

Various activation functions may be used. For illustration, both exponential linear units (ELU) and rectified linear units (ReLU) have been used successfully in the systems and methods disclosed herein. A wide variety of other activation functions may be used additionally or alternatively. These activation functions may include any one or any combination of Scaled Exponential Linear Unit (SELU), Gaussian Error Linear Unit (GELU) activation function, without being limited thereto. A wide variety of other artificial neural network layers may be used additionally or alternatively. These layers may include any one or any combination of fully connected layers; pooling layers (such as max-pooling); (self) attention modules; atrous convolutions; (batch) normalization layers; transformers; recurrent layers, such as long-short-term memory (LSTM), gated recurrent unit (GRU), without being limited thereto.

For even further enhanced efficiency and robustness, the AI model may be trained to provide information on the steering prediction in a reference frame that is spatially varying along the elongate curve. For illustration, the AI model may be trained (during a training phase) and may operate during inference to provide information on the steering prediction in a (local) Bishop frame, e.g., by outputting the two Bishop curvatures (see below for more details).

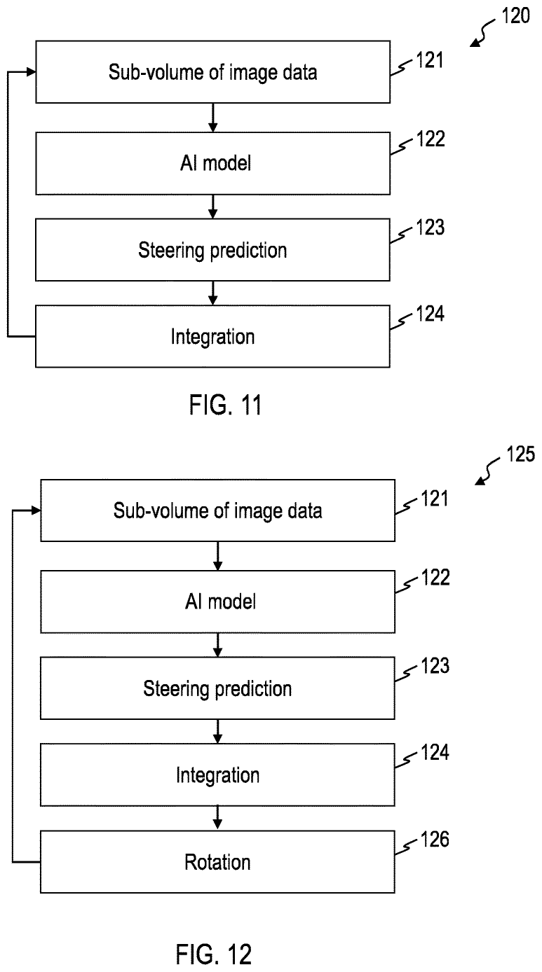

FIG. 11 is a flow chart of a method 120. The method 120 may be performed automatically by the image processing system 30. The method 120 may be performed to determine a series of points 60 along the center line 50 by recurrent inference.

At step 121, a sub-volume is selected for processing. The sub-volume may have a position that depends on a previously determined point along the center line (which may be specified by user input in the first iteration or as result of a preceding iteration in the later iterations). The sub-volume may have an orientation which depends on previous sub-volume orientations and previously determined steering predictions (which may be specified by user input or determined automatically in the first iteration and which may be taken from the preceding iteration in the later iterations).

At step 122, the AI model operates on the image data within the sub-volume. Interpolation of image data voxels may be performed to determine the voxel values in the voxel grid of the respective sub-volume, for arbitrary orientations and/or locations of the sub-volume.

At step 123, the AI model may provide, as AI model output, the updated steering prediction. The updated steering prediction may be specified relative to a local reference frame, e.g., as curvature values relative to a reference frame given in relation to the sub-volume (such as a Bishop coordinate system). Additional output may be provided by the AI model, such as information on a distance from an obstacle.

At step 124, a new point along the center line may be determined based on the steering prediction from step 123. For illustration, integration may be performed to determine the new point along the center line.

FIG. 12 is a flow chart of a method 125. The method 125 may be performed automatically by the image processing system 30. The method 125 may be performed to determine a series of points 60 along the center line 50 by recurrent inference. The method 125 may comprise steps 121-124 explained with reference to FIG. 11. The method 125 may further comprise the additional step 126 of determining a rotation around the momentary flight direction, which is used for specifying the sub-volume in the subsequent iteration. Exemplary rotations 95, 96 are illustrated in FIG. 6.

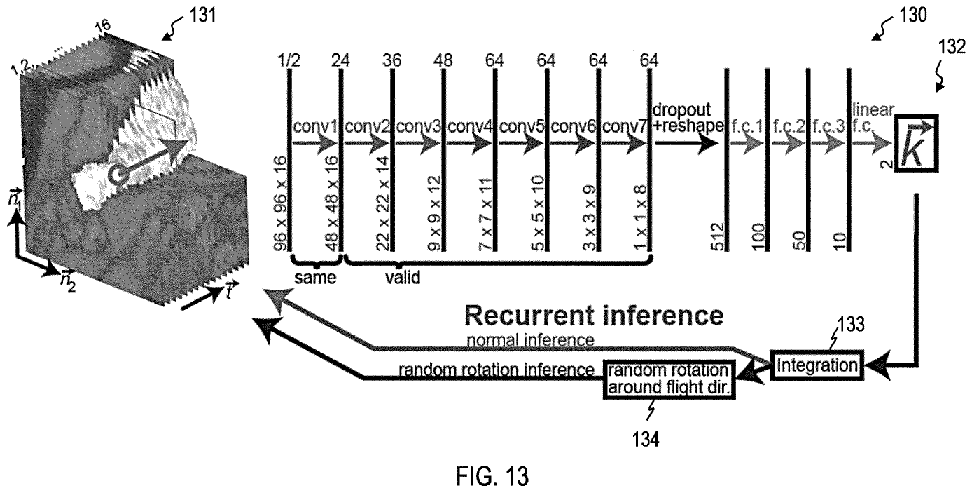

FIG. 13 illustrates an AI model architecture and data processing that may be employed in the methods and systems disclosed herein. In each iteration, a sub-volume 131 is analyzed that is centered (in directions perpendicular to the current flight direction) at the current position.

The AI model 130 may include an input layer operative to receive K×N×M voxel values for the image data in the sub-volume 131. The number of voxels K and N in the directions transverse to the most recent steering prediction may exceed the number of voxels M parallel to the most recent steering prediction. For illustration, K=N=96 and M=16 may be used. Additionally, the physical size of a single voxel for the sub-volume may be chosen anisotropically and independently of the physical size of a single voxel in the image data 40.

The values K and N may be set in dependence on the object that is being traced. For illustration, for an object known or expected to have an elliptical cross-section in a plane transverse to the center axis, it may be useful to set K and N to different values.

In the implementation of FIG. 13, the AI model comprises seven strided convolutional layers conv1-conv7. The layers do not all need to be the same. For illustration, three of the convolutional layers conv1-conv3 may have 5×5×3 convolution with stride 2×2×1, and four of the convolutional layers conv4-conv7 may have 3×3×2 convolution with stride 1×1×1.

In the implementation of FIG. 13, the AI model comprises a dropout layer, which may have a dropout rate of 0.5.

In the implementation of FIG. 13, the AI model comprises three fully connected layers f.c.1-f.c.3 and a linear layer.

The AI model output 132 may be the steering prediction, optionally determined in a local reference frame (e.g., as Bishop curvature).

The processing includes an integration 133 to determine, based on the AI model output 132, the next point along the center line. The sub-volume analyzed in the subsequent iteration may be set without additional random rotation (normal interference path in FIG. 13) or with additional random rotation 134.

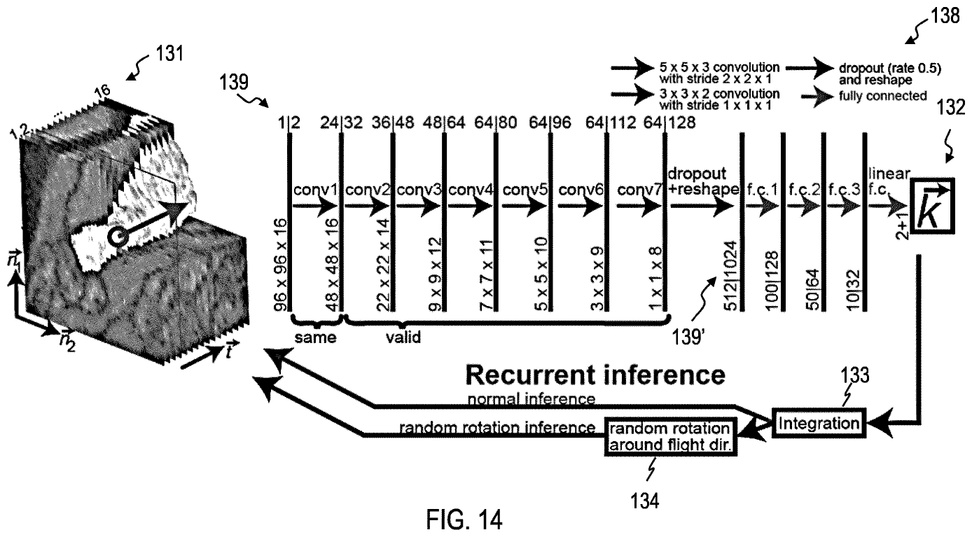

FIG. 14 shows a further implementation of the AI model 138 that may be used in the methods and systems disclosed herein. Parameters 139 of the convolutional layers conv1-conv7 may be modified as compared to the AI model of FIG. 13. Alternatively or additionally, parameters of the fully connected layers f.c. 1-3 may be modified as compared to the AI model of FIG. 13. For illustration, either the parameter values before the vertical bar or after the vertical bar indicated in FIG. 14 may be utilized. In one implementation, the parameter values 139 and the parameter values 139′ indicated behind the vertical bar in FIG. 14 may be used.

The systems and methods can be used for neurite tracing, without being limited thereto. The systems and methods allow neurite reconstruction to be performed from a 3D-EM volume as a center line reconstruction task, in which the neurite is represented by a sequence of visited points. The CNN is trained on the task of predicting the local neurite continuation from a neurite-centered and -aligned 3D-EM sub-volume, similar to human annotation flight mode see, e.g., Boergens et al. 2017. “WebKnossos: Efficient Online 3D Data Annotation for Connectomics.” Nature Methods 14 (7): 691-94).

Integration of the predicted neurite continuation yields a new position and orientation, which is used to generate the subsequent CNN input. Iterative application of this procedure turns a start location and orientation into a neurite skeleton reconstruction using only 3D-EM data and without intermediary steps, such as volume segmentation.

For the AI model of FIG. 13, the input to the CNN consists of a 96×96×16 Voxels (Vx) neurite-centered and -aligned 3D-EM sub-volume 131. The sub-volume may cover, e.g. for axons, a field of view of around ~1×1×0.7 μm (cf. FIG. 13).

The third (Z) dimension corresponds to the current flight direction of the process, while the current position can be made the center of the 4th Z-plane.

The field of view is thus asymmetric along the Z direction with more contextual information available in forward than in reverse flight direction. This allows for better steering towards the ‘exit’ within axonal varicosities.

The EM data may be projected onto axon-aligned planes by means of trilinear interpolation.

Note that only by using a 3D EM sub-volume as input it is possible to extract relevant information about the continuation of a neurite from a single input. Alternatively, one can also feed 2D EM images into a recurrent neural network that can internally keep track of the sequence of images and thereby estimate neurite continuation as well.

For the CNN architecture of FIG. 13, seven 3D strided convolutional layers were used, followed by a dropout layer (dropout rate: 0.5), three fully connected layers and a last linear layer that estimates the two steering commands and the distance to membrane. The two steering values output by the output layer as steering prediction may represent the Bishop curvatures described in more detail below. As nonlinearity ELU were used (see, e.g., D. A-. Clevert et al. 2015. “Fast and Accurate Deep Network Learning by Exponential Linear Units (ELUs).” Iclr. 566 https://doi.org/10.3233/978-1-61499-672-9-1760).

For the formulation of the network's input and output the Bishop frame and the associated Bishop curvatures may be used, as will be described in the Techniques section below. The Bishop frame is a local orthonormal coordinate system spanned by a tangential vector and two normal vectors and obeys a rotation minimizing property disallowing any twists around the tangential vector. The neurite aligned projection planes for the AI model's input may be defined by means of the Bishop normal vectors, while the network's flight direction may correspond to the tangential vector.

The evolution of the Bishop frame unit vectors is coupled via the Bishop curvatures, one for each normal vector direction.

The Bishop curvatures may be the output of the AI model output layer that is used for steering. Each of the two Bishop curvatures resembles a signed curvature for the corresponding normal vector direction. The signed curvature has also been used as steering command for image-based road following, albeit only for one steering direction corresponding to planar curves (see, e.g., KM. Bojarski et al. 2016. “End to End Learning for Self-Driving Cars.” ArXiv E-Prints. https://arxiv.org/pdf/1604.07316.pdf).

The task of predicting the neurite continuation in the form of Bishop curvatures can be interpreted as fitting a parabola to the neurite's center line. The curvature vector, calculated as the sum of Bishop curvatures multiplied by their corresponding Bishop normal vector, determines direction and magnitude of bending of this parabola.

The Bishop's curvatures output by the AI model output layer may, thus, be integrated to determine a next one of a series of points along the center line (with the series being a priori unknown and being determined to trace the center line).

### Training

Operation of the trained AI model has been explained above. Systems and methods disclosed herein may be operative to train the AI model for its intended use.

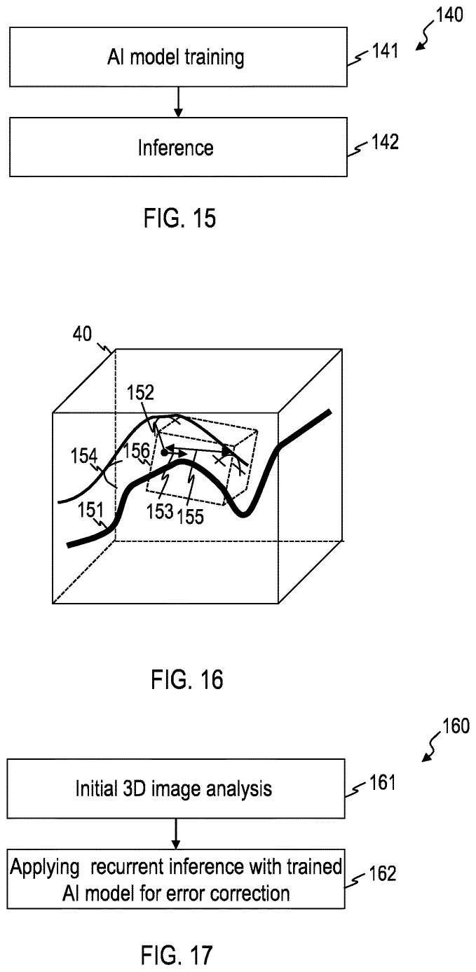

FIG. 15 is a flow chart of a method 140 that includes both AI model training 141 and operation of the trained AI model during inference 142. Use of the trained AI model at step 142 may be implemented using any one of the techniques for inferring a direction along which an elongate object extends described herein.

The training at step 141 may be a supervised training. Sets of annotated 3D-EM images may be used in the training. The training may comprise gradient-based updates of AI model parameters. Techniques known to the skilled person may be used for updating model parameters, such as gradient descent techniques.

The training at step 141 may comprise training the AI model to comply with a flight policy that causes the AI model to converge back to the center line 50 even when starting from or reaching positions off the center line. To accomplish this, the training step 141 may comprise training the AI model (also) on scenarios in which AI model starts from a position off the center line 50 and/or predictions of the AI model during training may be integrated and fed back as done in inference to potentially also yield positions off the center line. A flight policy may be implemented which enforces convergence back to the center line 50, even when the initial sub-volume (i) starts from a location that is offset from the center line and/or (ii) is not aligned with the direction of the elongate object. Thus, training may also be performed using sub-volumes in which none of the voxel planes of the sub-volume 91, 93 has a center that coincides with a location along the center line 50. At least a fraction of the sub-volumes may be deliberately chosen such that the center of, e.g., a 4th plane (out of 16 planes of the sub-volume for the model in FIG. 13) has an offset from the point on the center line determined by the annotated data or in the preceding iteration. The offset may be directed transversely to the previously determined extension direction (i.e., transversely to the steering prediction in the Bishop frame).

The training may be implemented in a manner which uses one or several training parameters that are variable during the training. This allows different policies to be enforced during training.

Results of the elongate object model training improve even further when using such training techniques.

In the training at step 141, a convergence distance of the flight policy may determine how rapidly the iterative process converges back to the center line. The convergence distance may be a dynamic parameter that is varied during AI model training. The convergence distance may be set as a function of a distance from a physiological boundary during AI model training. The convergence distance may be a monotonously increasing function of the distance from the physiological boundary, thereby implementing a more aggressive return to the center line at positions that are closer to a physiological boundary, such as a cell boundary.

Alternatively to supervised learning with a hand-crafted flight policy, the flight policy could also be learned directly using reinforcement learning by defining a reward function instead that encourages stable tracing of the elongate object.

As a specific example, the CNN architecture of FIG. 13 can be trained on a neurite following task using TensorFlow (M. Abadi et al. 2016. “TensorFlow: A System for Large-Scale Machine Learning.” In 12th USENIX Symposium on Operating Systems Design and Implementation (OSDI '16), 265-537 84). The AI model can be trained with a mini-batch size of 128 using RMSProp (see, e.g., T. Tieleman and G. Hinton. 2015. “Neural Networks for Machine Learning Lecture 6a: Overview of Mini-batch Gradient Descent.” Lecture Notes. 2015. http://www.cs.toronto.edu/~tijmen/csc321/slides/lecture_slides_lec6.pdf) with momentum (D. E. Rumelhart et al. 1986. “Learning Representations by Back-Propagating Errors.” Nature 323 (6088): 533-36; I. Sutskever et al. 2013. “On the Importance of Initialization and Momentum in Deep Learning.” In International Conference on Machine Learning, 1139-1147) or Adam (D. P. Kingma and J. L. Ba. 2015. “Adam: A Method for Stochastic Optimization.” In 3rd International Conference on Learning Representations, ICLR 2015-Conference 607 Track Proceedings. https://arxiv.org/abs/1412.6980) on minimizing the mean squared error plus a L2 regularization loss term on the neural network training parameters.

Training and subsequent testing has shown that performance during inference can be enhanced by training also on positions that are off the center line. During inference current position and orientation depend on past AI model decisions. Therefore, even small errors in past network's steering predictions can lead the network away from the neurite center line.

To enhance stability of path following (i.e., tracing), the AI model may also be trained on off-center positions and off directions with correspondingly adjusted steering predictions leading back to the neurite center line. To sample off-center positions within the neurite, the neurite diameter and/or shape has to be known at least approximately during training. A volumetric reconstruction of the neurite can be used to determine off-center positions within the neurite. The mapping from a particular off position, off direction state to the adapted steering is also referred to as flight policy.

A greedy flight policy may be used that is only based on local information available to the AI model within the finite field of view, as described in more detail herein. The distance within which to converge back to the center line is a free parameter of this flight policy. Different convergence distances may be used for off-center positions within a synaptic bouton. When closer to ‘hitting’ the cell membrane, a more aggressive steering with shorter convergence distance is useful to stay within the neurite, while for larger distances to the membrane a gentler steering is affordable and better preserves neurite alignment of the input field of view. Thus, a dynamic convergence distance may be used that may be set in dependence on (e.g., equal to) the distance to the cell membrane along the flight direction during training. This induces the notion of an obstacle, i.e. cell membrane, avoidance and empirically performs better than use of a constant value for the convergence distance during training.

FIG. 16 shows a volume 40 of 3D training data. A neurite 151 has been annotated in the 3D training data. The AI model is trained using also sub-volumes such as exemplary sub-volume 156 around points such as exemplary point 152, with the exemplary point 152 (that is located in the center of one voxel plane of the sub-volume 156) being offset from the center line of the neurite 151. The flight direction 153 may be (significantly) different from the extension direction of the neurite within sub-volume 156 during training. One or several training parameters, such as a convergence distance that dictates how quickly the process is to converge back to the center line of the neurite, may be dynamically varied. For illustration, the convergence distance may be set in dependence on a distance 155 from a cell membrane 154 which is shown merely schematically. The distance 155 may be measured along the momentary flight direction.

### Performance with Regard to Error Rates

To assess performance, the image processing systems and methods are evaluated on a validation set and two test sets of linear axonal branches by running recurrent inference starting from both sides. Reaching a set of thresholds concerning the distance or the angle with respect to ground truth tracing triggers a reset to the ground truth tracing, as disclosed in more detail herein. Tracing is stopped when the network reaches the other end of the axonal branch.

Each steering error leading to a reset corresponds to both a merge error into a wrong process and a split error due to not continuing the neurite of interest. Each reset is counted as two errors. The error rate is the number of errors divided by the path length of the ground truth skeletons. This metric allows for model selection in case of multiple trained AI models based on a validation set, as well as a rough comparison with other automated approaches, for which the inter-error distance, i.e. the inverse error rate, is reported, as well as for comparison to human error rates.

The image processing systems and methods were trained on a 3D-EM dataset from mouse primary somatosensory cortex layer 4 acquired using serial block-face scanning electron microscopy (SBEM, (W. Denk and H. Horstmann. 2004. “Serial Block-Face Scanning Electron Microscopy to Reconstruct Three-Dimensional Tissue Nanostructure.” Edited by Kristen M. Harris. PLOS Biology 2 (11))). The dataset has a size of 61.8×94.8×92.6 μm³ and a voxel size of 11.24×11.24×28 nm³.

For model selection, the image processing systems and methods were evaluated on a validation set consisting of 13 axons with 1.4 mm path length. For the validation set axonal branches with less than 5 μm path length were excluded for a better heuristic detection of errors. Training and validation set axons are a disjoint random subset of axons seeded by means of presynaptically classified segments obtained using the techniques disclosed in B. Staffler et al. 2017. “SynEM, Automated Synapse Detection for Connectomics.” ELife 6 (July): e26414. Averaged over all models and training iterations the random inference mode outperforms its normal mode counterpart by 33% (range: 12-52%).

An AI model that was identified to have particularly good performance on the validation set uses only EM data as input, has an ELU activation function, was trained for 700,000 training iterations and yields, based on heuristic error detection, 16.4 errors/mm. The manual inspection of tracings of the inventive technique yields 1 false positive and 0 false negative resets (precision 96%, recall 100%). The true error rate is 15.7 errors/mm.

Application of the selected AI model on a set of 59 soma seeded axons with 6.2 mm path length yields 29.8 errors/mm at 185 resets with 9 false negative resets and 5 false positive resets (precision 95%, recall 95%). The true error rate is 30.4 errors/mm.

Knowing that soma seeded axons are biased towards larger diameters, a test was also performed for a random set of 10 axons with 1.6 mm path length previously used for the quantification of the human error rate (see, e.g., Boergens et al. 2017. “WebKnossos: Efficient Online 3D Data Annotation for Connectomics.” Nature Methods 14 (7): 691-94.). On this test set the image processing systems and methods disclosed herein yielded 58.1 errors/mm via the heuristic error detection. Manual inspection reveals 4 false positive and 2 false negative resets (precision 96%, recall 98%) leading to a true error rate of 55.6 errors/mm.

### Possible Further Training and Test Implementations

Exemplary training data and evaluation techniques were already described above. Possible further 3D-EM datasets of neural tissue and within these training data, validation and test sets that can be used in association with the methods and systems disclosed herein are provided next.

The methods and systems may be trained and/or used on a 92.6×61.8×94.8 μm³ SBEM dataset (W. Denk and H. Horstmann (2004). Serial block-face scanning electron microscopy to reconstruct three-dimensional tissue nanostructure. PLOS Biology, 2, e329. doi: 10.1371/journal.pbio.0020329) from layer 4 primary somatosensory cortex of a 28 days old mouse previously densely reconstructed by A. Motta et al. (2019). Dense connectomic reconstruction in layer 4 of the somatosensory cortex. Science, eaay3134. doi: 10.1126/science.aay3134. The tissue was conventionally en-bloc stained (K. L. Briggman et al. (2011). Wiring specificity in the direction-selectivity circuit of the retina. Nature, 471, 183-188. doi: 10.1038/nature09818) and imaged at 11.24×11.24 nm² and nominal cutting thickness of 28 nm. Training and validation axons may be sampled from a set of axons seeded by means of presynaptically classified segments obtained using SynEM (B. Staffler et al. (2017). SynEM, automated synapse detection for connectomics. Elife, 6, e26414. doi: 10.7554/eLife.26414) and skeleton traced by annotators. To also acquire a volume reconstruction of training axons, segments of the oversegmentation obtained using SegEM with parameters as set for the whole-cell segmentation of the cortex dataset (M. Berning et al. (2015). SegEM: Efficient Image Analysis for High-Resolution Connectomics. Neuron, 87, 1193-1206. doi: 10.1016/j.neuron.2015.09.003) may be picked up and combined. The volume mask may also be used to iteratively optimize the interpolated skeleton tracing to yield a better centerline approximation. A set of 14 axons with 1.2 mm path length may be used for training, and up to 700,000 weight updates corresponding to ~260 epochs were run. The validation set may consist of 13 axons with 1.4 mm path length, where branches with less than 5 μm were excluded for better heuristic error detection. A third set of 10 axons with 1.7 mm path length seeded from a (2.5 μm)³ bounding box may be used as a test set. These are the same axons as previously used to evaluate human and semi-automated segmentations (K. M. Boergens et al. (2017). webKnossos: efficient online 3D data annotation for connectomics. Nature Methods, 14, 691-694. doi: 10.1038/nmeth.4331; Motta et al., 2019, loc. cit.).

For automated spine head attachment on this dataset the axon-trained AI model (as shown in, e.g., FIG. 14 and as explained in more detail herein) may be evaluated on a random subset of 50 spine heads previously attached by human annotators, as well as on the set of spine heads previously used as test set in Motta et al., 2019, loc. cit.

Next, the AI model of the method and systems disclosed herein may be trained, tested and applied on a subset of a 1.3×1.3×0.25 mm³ dataset from a barrel cortex of a 28 days old mouse that may be stained following the protocol by Y. Hua et al. (2015). Large-volume en-bloc staining for electron microscopy-based connectomics. Nature Communications, 6, 7923. doi: 10.1038/ncomms8923 with small modifications, sectioned at 35 nm using ATUM (K. J. Hayworth et al. (2006). Automating the collection of ultrathin serial sections for large volume TEM reconstructions. Microscopy and Microanalysis: Cambridge University Press.) and imaged at 4×4 nm² with a multibeam scanning electron microscope (A. L. Eberle et al. (2015). High-resolution, high-throughput imaging with a multibeam scanning electron microscope. Journal of Microscopy, 259, 114-120. doi: 10.1111/jmi.12224).

The segmentation and agglomeration that may be applied to the dataset at a resolution of 8×8×35 nm³ (downsampled in x-y by a factor of 2) can be performed using, e.g., conventional approaches or techniques based thereon: In brief, a 3D U-Net (O. Ronneberger et al. (2015). U-net: Convolutional networks for biomedical image segmentation. Lecture Notes in Computer Science (including subseries Lecture Notes in Artificial Intelligence and Lecture Notes in Bioinformatics): Springer, Cham.) may be used to predict per cardinal axis voxel affinities following Lee, K., Zung, J., Li, P., Jain, V., & Seung, H. S. (2017). Superhuman Accuracy on the SNEMI3D Connectomics Challenge. arXiv e-prints, from which a watershed-based oversegmentation was generated. The segments from the oversegmentation may then be combined using hierarchical agglomeration as proposed in J. Funke et al. (2019). Large Scale Image Segmentation with Structured Loss Based Deep Learning for Connectome Reconstruction. IEEE Trans Pattern Anal Mach Intell, 41(7), 1669-1680. doi: 10.1109/TPAMI.2018.2835450. In addition, neurite type predictions, blood vessel and nuclei detection may be incorporated into the agglomeration to further reduce merge errors. Neurite type predictions were also used for spine head detection, which was the basis for spine head attachment based on the methods and systems disclosed herein.

For training of the AI model of methods and systems disclosed herein on axons, a training set may be acquired based on a set of 10 soma seeded axons from Layer 4, for which annotators may pick up segments from the oversegmentation to acquire a volume reconstruction. Kimimaro (W. Silversmith et al. (2021). Kimimaro: Skeletonize densely labeled 3D image segmentations. In GitHub repository (v 3.0.0 ed.)) may be used followed by subsampling and B-spline interpolation to extract centerline skeletons from the volume reconstruction. This can yield a training set with a total of 21 mm axon path length. As axon validation set a random 10 out of 20 axons from a Layer 4 seeded bounding box of size (2 μm)³ may be traced within a (50 μm)³ bounding box yielding 1 mm path length. For the axon test set, another (1.5 μm)³ bounding box within layer 4 was densely annotated and a random subset of 5 axons may be traced within the (150 μm)³ subvolume Si150L4 (accessible at https://wklink.org/7122) yielding 1.7 mm path length.

A separate AI model according to methods and systems disclosed herein may be trained on spine attachment. For this training and validation set may be generated from a set of 20 (5 μm)³ bounding boxes sampled within layer 4 and annotated for spine heads. A subset of around 1000 spine heads may be volume annotated at 4×4×35 nm³ by annotators from the spine head through the spine neck up to the dendritic trunk. Here, we again used Kimimaro (W. Silversmith et al., 2021, loc. cit.) to extract centerline skeletons from the volumetric masks for training yielding 2 mm of spine neck tracings. Additionally, a random subset of 76 spine heads with 0.2 mm path length from the 20 bounding boxes may be skeleton traced to serve as validation set. The evaluation may be done on another randomly selected (5 μm)³ bounding box (Si11L3, accessible at https://wklink.org/2458) containing 91 densely annotated spine head to dendritic trunk skeleton tracings.

For the evaluation of an error correction of another state-of-the-art segmentation that can be performed using the methods and systems disclosed herein, the methods and systems disclosed herein may be applied to a recently published mm³-scale multiSEM dataset with voxel size 4×4×33 nm³ (A. Shapson-Coe et al. (2021). A connectomic study of a petascale cerebral cortex. bioRxiv, 2021.2005.2029.446289. fragment of human doi: 10.1101/2021.05.29.446289) segmented and agglomerated using flood-filling networks (FFN; M. Januszewski et al. (2018). High-precision automated reconstruction of neurons with flood-filling networks. Nature Methods. doi: 10.1038/s41592-018-0049-4). Specifically, the training and evaluation of the techniques may focus on a (150 μm)³ bounding box containing 6.5 mm of axon path length of the provided ground truth skeleton tracings. The published ground truth skeleton tracings in this box may be used to evaluate FFN on soma-seeded axons. To finetune the processing by the methods and systems disclosed herein on this dataset, ground truth skeleton tracings of dense seeded axons may be generated by sampling a bounding box of size (2.5 μm)³ within a centered (15 μm)³ bounding box, annotating all processes in this bounding box and then sampling a random subset of 5 axons, which may be traced throughout the (150 μm)³ bounding box yielding 1.25 mm path length. Here, skeleton annotations were already done with high precision along the centerline such that no postprocessing was necessary. The volumetric neurite mask needed for training may be generated by means of segment pick-up from the c3 FFN segmentation, cf. (Shapson-Coe et al., 2021, loc. cit.). The best model checkpoint for the model used by the methods and systems according to embodiments, based on the validation set from the axon training in the mouse cortex ATUM-multiSEM dataset, was then used as initialization. The model may be trained for another approximately two million gradient updates until converging in terms of reset-based error rates on the training axons.

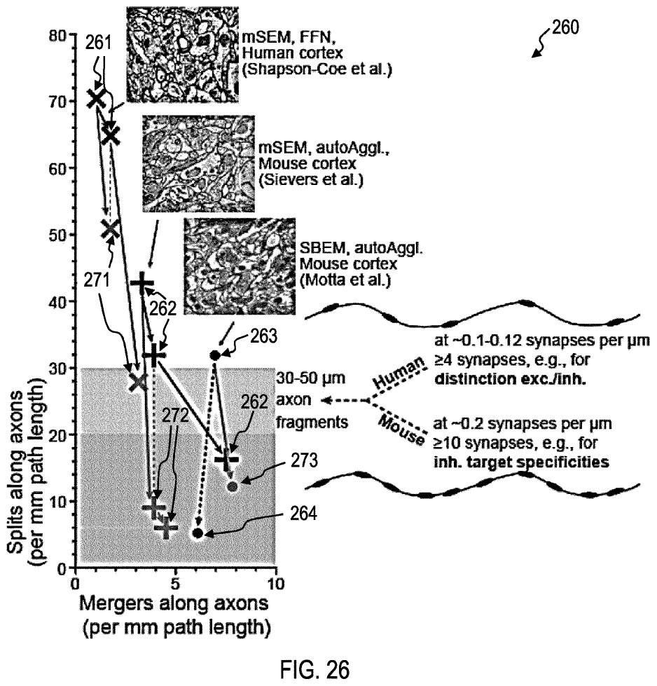

For the evaluation of FFN and the methods and systems disclosed herein (see, e.g., FIG. 26 and accompanying description), another bounding box of size (1.5 μm)³ may be annotated for a random subset of 5 axons traced throughout the (150 μm)³ bounding box yielding 1.4 mm path length.

Further, the methods and systems disclosed herein may be trained or used on any or a combination of
artificially generated data mimicking the appearance of elongate objects to be traced during inference
X-ray, specifically X-ray holographic nano-tomography, light-microscopy, or magnetic resonance imagery of one or many elongate objects.

The required amount of training data in path length for the methods and systems disclosed herein to work as described may in general depend among others on the resolution and visual complexity of the data, the variability in morphology and appearance of elongate objects to be traced, the algorithms and artificial neural network architectures employed for training, the number, kinds, and order of image data used for training.

### Performance Compared to Other Image Processing Approaches

Performance of the image processing systems and methods disclosed herein was also compared to other techniques.

It was investigated to what degree techniques disclosed herein can replace human annotation, and which kinds of connectomic analyses would thus become fully automatable. For this we started with the dense connectomic analysis of a piece of mammalian neocortex (A. Motta et al. 2019. “Dense Connectomic Reconstruction in Layer 4 of the Somatosensory Cortex.” Science. Vol 366. Issue 6469. p. eaay3134) in which based on automated segmentations, human annotators had been asked to resolve an automatically identified set of problem locations consuming a total of 4,000 work hours. To resolve split errors, human queries had been placed at endings of objects, asking to continue if possible, and stopping the task when another automatically reconstructed object had been reached. To resolve merger errors, chiasmatic configurations had been detected, and queries had been placed at the exits, asking for proper continuation into one of the other chiasmatic exits. Techniques disclosed herein were used to replace these human annotations by starting techniques disclosed herein queries similarly to the human annotator queries. In fact, 76% of the ending tasks and 78% of the chiasma tasks were correctly traced by techniques disclosed herein (Human: 74% and 94% respectively). Additionally, running techniques disclosed herein from the endpoint back to the start gives an error signal allowing to avoid most merge errors yielding only 4% of queries with new techniques disclosed herein introduced merge errors for ending tasks and 1% for chiasma tasks, comparable to human annotations.

While this performance indicated that techniques disclosed herein could faithfully replace human annotation, we wanted to quantify this using connectomic analysis directly as the metric for reconstruction success. Some connectomic analyses require more reconstruction precision than others. In particular, the following were considered: (1) paired same axon-same dendrite synapse analyses aimed at measuring the learned fraction of a connectome (Motta et al., loc. cit.); (2) spine rate analyses for identification of interneuron dendrites; (3) axonal type analysis based on the synaptic target distribution of axons. Three types of connectomes were used for comparison: (I) the connectome obtained from the fully automated reconstruction in (Motta et al., loc. cit.), before any focused human annotation; (II) the connectome in (Motta et al., loc. cit.) including 4,000 work hours of human annotation; (III) the connectome obtained from combining the fully automated reconstruction in (Motta et al., loc. cit.) with techniques disclosed herein, yielding a fully automated and automatically proofread connectome. These three types of connectomic analyses (I), (II), and (III) were performed. While the paired synapse analysis yields similar results already with the automated state before corrections attained by inventive techniques, correct spine rates for apical dendrites and the true fraction of excitatory axons defined by their spine head preference require human corrections or corrections attained by inventive techniques. Additionally, corrections attained by inventive techniques recover the axonal target specificities of inhibitory axons onto apical and smooth dendrites, whereas in the automated state prior to techniques disclosed herein and human corrections this specificity was not detectable. So in fact, when utilizing a connectomic metric of automation qualified by the type of connectomic problems that can be automatically analyzed, error correction using techniques disclosed herein-shifts the automated analysis performance from simpler to more complex connectomic problems.

The techniques disclosed herein were also applied to datasets from the Automatic Tape-collecting Ultra-Microtome (ATUM)/multiSEM imaging approach that are substantially larger and of different image resolution and contrast. The useability of techniques disclosed herein was tested for seeded axonal reconstructions: reconstruction of axons starting at the cell body. After initial segmentation and agglomeration steps, endings were queried using techniques disclosed herein in the following way: axonal endings were detected and techniques disclosed herein traced these until a new agglomerate of 2.5 μm length was found or any of a number of stopping criteria was met. To avoid merge errors, new agglomerates were only added if techniques disclosed herein validated the tracing in backwards direction and a number of type and angle criteria were met. A second inventive tracer trained on spine necks was used if the axon-trained techniques disclosed herein failed. Performing 20 iterations per axon, it was found that techniques disclosed herein were able to extend axonal recall from 7% to 37% (median: from 6% to 44%; precision decreased from >99% to 74%), making local connectomic analysis possible in volumes sized about 200-300 μm on a side, corresponding to local intra-layer connectivity of mouse cortex.

Similar to thin axons, thin spine necks can pose difficulties for current reconstruction pipelines. However, to assign synapses onto spine heads to the correct cell, spine necks need to be reconstructed with high precision. Thus, it was tested whether techniques disclosed herein can improve spine head attachment precision/recall from a global agglomeration (100%/43%) and a local agglomeration (100%/75%; similar strategy as in (Motta et al., loc. cit.). To this end, techniques disclosed herein starts tracing in detected spine heads for two candidate directions with lowest uncertainty in the prediction. Inventive techniques disclosed herein trace for a maximum of 7 μm or until reaching a dendrite agglomerate of sufficient size. Order of candidates, type and attachment information is then used to add the spine head to the dendrite agglomerate. This boosts spine head attachment recall from 75% to 94%, while retaining high precision of 97%.

The inventive techniques also provide enhancements as compared to techniques such as Flood-Filling Networks (M. Januszewski et al. 2018. “High-Precision Automated Reconstruction of Neurons with Flood-Filling Networks.” Nature Methods. https://doi.org/10.1038/s41592-018-0049-4). Application of the inventive techniques on automatically detected endings from a Flood-Filling Networks (FFN) segmentation solves 60% of the splits on a random set of axons traced throughout the sub-volume thereby reducing the split rate from 65-70 splits/mm to 28-31 splits/mm, while only introducing few new merge errors (merge rate increased from 3-4 mergers/mm to 5-6 mergers/mm). Notably, evaluation on random axons not necessarily connected to a soma within the mm³-scale volume gives a better picture of the reconstruction quality than a restriction to soma-proximal axons. Evaluation of FFN restricted to axons connected to a soma within the volume, as previously used to showcase reconstruction performance (Januszewski et al., 2018) underestimates split and merge errors of random axons 3-4-fold (15-22 splits/mm, 0.2-1.1 merger/mm).

As shown by these results, the techniques disclosed herein may be used as fully automatic annotation techniques for a wide variety of cases and yields sufficiently orthogonal data that can improve all currently available automated segmentations. This was in particular notable for segmentations obtained using the current state-of-the-art technique, flood filling networks (FNNs). The computational complexity of the inventive techniques disclosed herein are favorably lower than other approaches, and with the direct end-to-end strategy of inventive techniques may allow optimization for both additional accuracy and computational efficiency, which will be the next key challenge in connectomics for exabyte-scale datasets to come (A. Motta et al. 2019. “Big data in nanoscale connectomics, and the greed for training labels”. CurrOpNeurobiol. Volume 55, April 2019, Pages 180-187).

### Efficiency Compared to Other Image Processing Approaches

To estimate computational costs, we benchmarked the inventive techniques disclosed herein on ~128,000 ending tasks from the first set of ending detections in (A. Motta et al. 2019. “Dense Connectomic Reconstruction in Layer 4 of the Somatosensory Cortex.” Science. Vol 366. Issue 6469. p. eaay3134). The step size factor was set to ƒ=5 and analytically derived equations for the Bishop frame along a parabola were used to integrate steering predictions. The total runtime on a single node using 32 cores (Intel® Xeon® Gold 6130 CPU, 2 sockets), a single Tesla V100 GPU (PCIE-16 GB) and less than 128 GB RAM was 13.6 hours for the reconstruction of around 2.1 meters of axons (including backward validation tracings, a total of 64 million CNN inferences), and hence a reconstruction speed of around 160 mm/h (~1300 steps/s, average step size of 33 nm). From this and previous runs of the inventive technique with ƒ=1 and integration using the forward Euler method, we extrapolated to yield a total runtime of 27.9 hours for axon and spine neck tracing (4.3 meters), and 7.6 hours for initial direction prediction (on ~138,000 spine heads) on a single node for the automated error correction of the reconstruction state from (Motta et al., 2019) before human interventions yielding a total of 35 single GPU node hours. For Flood-Filling Networks the 6.964 GPU node hours were multiplied by the 1000 (NVIDIA P100) GPUs (Januszewski et al., 2018). For Local Shape Descriptors the AcRLSD architecture (Sheridan et al 2021. “Local Shape Descriptors for Neuron Segmentation.” bioRxiv, 2021.01.18.427039v1. doi: 10.1101/2021.01.18.427039v1) took a total of 10.5 hours on 24 NVIDIA V100 GPU nodes, watershed and agglomeration took 7.7 CPU node hours with 100 cores. For (Motta et al., 2019), the dense reconstruction including segmentation, agglomeration, type and synapse prediction and processing of human skeleton reconstructions took 101 hours on 24 nodes with 16 CPU cores each. For all methods the CPU and GPU hours were multiplied by the ratio of the respective dataset sizes (Motta et al., 2019), resulting in the compute cost estimates provided in the Table below.

TABLE
Compute cost estimates

| | | | CPU | GPU | Dataset | Compute |
|---|---|---|---|---|---|---|
| | | | core | node | size | cost |
| Approach | Hardware | Runtime | hours | hours | factor | estimates |
| FFN | 1000x NVIDIA Tesla | 6.964 h | — | 6964 | 0.5 | 3482 GPU |
| (Januszewski et al., | P100 nodes | | | | | hours |
| 2018) | | | | | | |
| Dense reconstruction | 24x CPU nodes with | 101 h | 38.8 k | | 1 | 38800 |
| (Motta et al., 2019) | 16 CPU cores each | | | | | CPU core |
| | @ 16 GB RAM/core | | | | | hours |
| LSD (AcRLSD) | 24x NVIDIA Tesla | 10.5 h | — | 252 | 0.7 | 176.4 |
| (Sheridan et al., | V100 nodes for CNN | | | | | GPU |
| 2021) | inference | | | | | hours |
| | (3.808 USD/h/V100) | | | | | |
| | 100 CPU cores for | 7.7 h | 770 | — | | 539 CPU |
| | watershed and | | | | | core |
| | agglomeration | | | | | hours |
| Inventive technique | 1x NVIDIA Tesla | 35 h | — | 35 | 1 | 35 GPU |
| disclosed herein | V100 node | | | | | hours |
| | with 32 CPU cores | | | | | |
| | @ 128 GB RAM | | | | | |

### Operation of the Image Processing Systems and Methods in Combination with Other Techniques (e.g., as Error Corrector)

The processing of 3D EM data or other volumetric image data disclosed herein, which uses the trained AI model, can be combined with other reconstruction techniques. For illustration, the techniques disclosed herein may be used for performing connectomic reconstructions (or other 3D elongate object tracing) to replace human annotations in association with conventional annotation or tracing techniques. The techniques disclosed herein enable a fully-automated connectomic reconstruction of relevant volumes when used as an error corrector.

In embodiments, the image processing systems and methods disclosed herein may be used in association with other reconstruction techniques. For illustration,
an automated reconstruction process may be run,
responsive to detecting a missing continuation of an automated reconstruction, the iterative technique disclosed herein may be started at that location and may be run until it finds another automatically reconstructed component. From that point, and
optionally, responsive to finding another automatically reconstructed component, the iterative technique disclosed herein may be run in a backward direction to reconfirm that it ends up at the same start location (for verification purposes), and
the skeleton reconstruction of above defined procedure may be used to error-correct the automated reconstruction, optionally in an iterative manner.
   Further, for illustration,
an automated reconstruction process may be run,
responsive to detecting an incorrect continuation or merge error of an automated reconstruction, the iterative technique disclosed herein may be started at several locations around the error and may be run until it leaves a bounding box around the error location, and
optionally, responsive to leaving the bounding box around the error location, the iterative technique disclosed herein may be run in a backward direction to reconfirm that it ends up at the same start location (for verification purposes), and
the skeleton reconstructions of above defined procedure may be used to error-correct the automated reconstruction, optionally in an iterative manner and further optionally interleaved with iterations of detection and correction of missing continuations.

FIG. 17 is a flow chart of a method. The method may be performed automatically by the image processing system.

At step 161, an initial 3D image analysis is performed. The initial 3D image analysis may be performed for obtaining a 3D connectomic reconstruction. Techniques such as those described in A. Motta et al. 2019. “Dense Connectomic Reconstruction in Layer 4 of the Somatosensory Cortex.” Science. Vol 366. Issue 6469. p. eaay3134 or M. Januszewski et al. 2018. “High-Precision Automated Reconstruction of Neurons with Flood-Filling Networks.” Nature Methods. https://doi.org/10.1038/s41592-018-0049-4 without being limited thereto may be used to implement step 161.

At step 162, the image processing systems or methods disclosed herein may be used for acting as an error corrector. The image processing systems or methods may be employed for neurite (e.g., axon) tracing in cases where other automated techniques are incapable of annotating the image data.

For illustration, the techniques disclosed herein were applied to all detected spine heads determined using the techniques of A. Motta et al. 2019. “Dense Connectomic Reconstruction in Layer 4 of the Somatosensory Cortex.” Science. Vol 366. Issue 6469. p. eaay3134 that previously required human annotations (38% of all detections consuming around 900 working hours of annotators). A precision/recall of attached spine heads of 89%/84% was achieved, approaching a precision of human decision. The start positions from the spine head detection that were also provided to human annotators were used. The direction prediction of the techniques disclosed herein was then performed. The recurrent inference mode of the techniques disclosed herein were applied for the candidate direction with lowest prediction uncertainty and tracing was performed until a dendritic trunk defined by a dendrite mask was reached or a path length threshold was exceeded. Notably the precision/recall reported above was achieved without retraining the techniques disclosed herein on spine necks. Even further improvements can be made by training on a dedicated training set from spine necks.

For the correction of split and merge errors in the axon reconstruction, which previously consumed thousands of working hours of annotators, start positions and directions from the techniques of A. Motta et al. 2019. “Dense Connectomic Reconstruction in Layer 4 of the Somatosensory Cortex.” Science. Vol 366. Issue 6469. p. eaay3134 were supplied to the methods and systems disclosed herein. For split error resolution, the techniques disclosed herein were applied to small stretches and each stretch was validated by running the method backwards. Only if the validation was successful, the tracing continued with the next stretch until a known axon or the end of the dataset was reached. The resulting skeleton tracings could then be used analogous to human annotations. For merge error resolution, the stop criterion was based on a bounding box around the merge error-again analogous to human- and tracings were accepted if validation in backward direction yielded the same skeleton reconstruction with some error tolerance. Split and merge error resolution was run iteratively until remaining errors dropped below a threshold. The final axon reconstruction for this fully automated approach yielded 12 split errors/mm and 16 merge errors/mm on a representative set of 10 axons (1.7 mm), which is comparable to and approaches the error rate of human annotations (5 split errors/mm and 12 merge errors/mm) and outperforms conventional automatic techniques (Motta et al 2019 prior to human annotations: 32 split errors/mm and 14 merge errors/mm).

### Direction Prediction

As discussed herein, the image processing systems and methods are operative to trace an elongate object by iteratively determining the local direction (referred to as flight direction) along which an elongate object extends.

An initial direction (i.e., a direction that is used for selecting a sub-volume for analysis by the AI model in the first iteration) may be specified by user input. The image processing system may be operative to control the GUI to enable a respective user input for subsequent use by the elongate object tracing technique disclosed herein. Alternatively, when the initial direction is not obtainable in this way and is otherwise not known a priori, the initial direction may be set as a random direction (with the AI model typically guiding the process back to the center line in view of its training). Alternatively, a more targeted approach may be used in which Monte-Carlo dropout is used on a number of candidate orientations. The implementation of the direction selection from the candidates may be dependent on the stability as estimated by the image processing systems and methods. More robust predictions (i.e., direction predictions with smaller uncertainties) are given preference over predictions with higher uncertainty in the selection process.

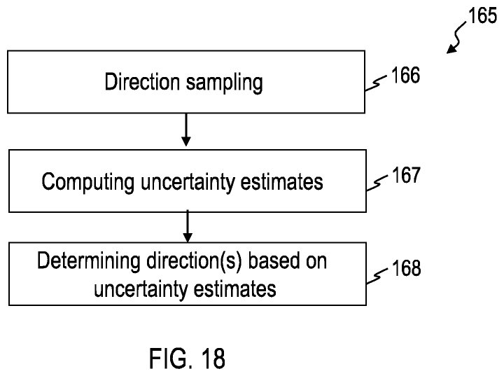

FIG. 18 is a flow chart of a method 165. The method 165 may be performed automatically by the image processing system or method.

At step 166, direction sampling is performed. A set of directions, which may be equidistant or roughly equidistant on the surface of a unit sphere, may be sampled.

At step 167, uncertainty estimates may be computed for the various directions. The computation of the uncertainty estimates may be performed in various ways. For illustration, the uncertainty estimates may be determined for a plurality of directions using a process that comprise running Monte-Carlo dropout, computing covariances of direction estimates (such as Bishop curvatures) per orientation, and determining the uncertainty estimate based on a covariance matrix of the direction estimates.

At step 168, one or several directions (such as an initial direction of the recurrent inference) may be determined based on the uncertainty estimates.

### Operation & Effects

The image processing systems and methods address the need for techniques capable of performing automated axon tracing (while lending themselves to other applications as well).

The image processing systems and methods address axon reconstruction in a manner similar to a flight reconstruction task, yielding a skeleton, by means of a sequence of visited points, rather than a volumetric (voxel) reconstruction. The image processing systems and methods provide an automated approach for predicting skeleton reconstructions in the field of connectomics directly from raw EM data.

The image processing systems and methods provide the possibility for an end to end optimization. The image processing systems and methods focus on the essence of neurite reconstruction: following neurites over long distances through the dataset.

The image processing systems and methods make use of the fact that neurite reconstructions and error metrics on the level of skeletons are more robust than their voxel-based counterparts, which suffer from ambiguities in the affinity of individual voxels, e.g. in the presence of staining or imaging artifacts.

The image processing systems and methods provide a new approach for neurite tracing, which is skeleton rather than voxel based and show good results.

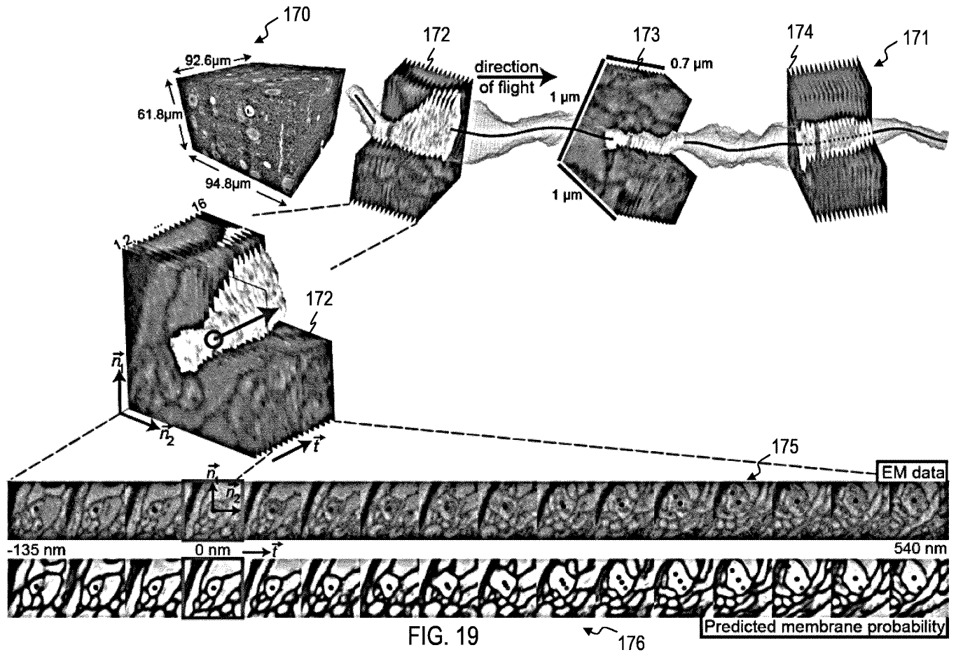

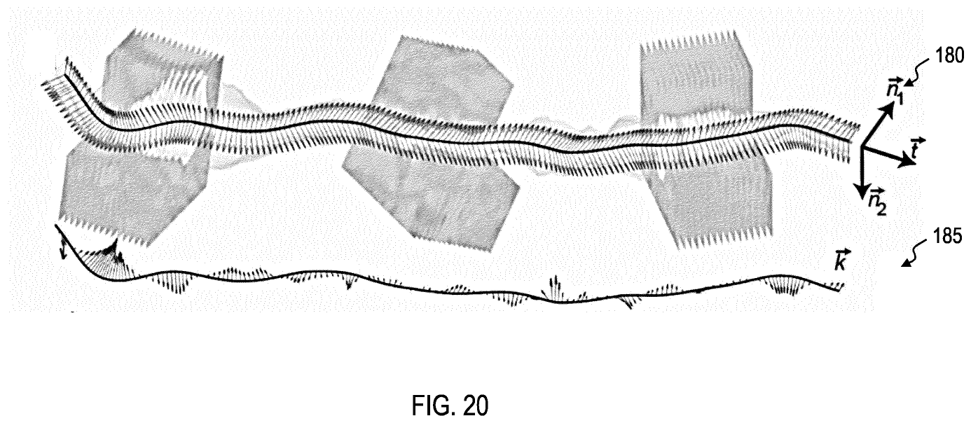

FIGS. 19 and 20 show operation of the image processing systems and methods. The image processing systems and methods are operative to trace neurites (e.g., axons) through a 3D volume represented by image data. Tracing here means determining a series of points in 3D or determining a continuous curve in 3D that specify the trace of the neurite in 3D. A series 171 of sub-volumes 172-274 is analyzed using the techniques disclosed herein. A number of planes 175 spaced along a flight direction (e.g., 16 planes) each include image data that is determined from the original image data using techniques such as interpolation or projection (e.g., trilinear projection). Information such as distance from a membrane may be determined and used in, e.g., AI model training. Additionally, membrane probabilities 176 as predicted by other methods, may be fed as input to the AI model, however in experiments this only helped in the initial training phase, while ultimately the raw EM data 175 provides enough information about neurite continuation for the method to be successful.

FIG. 20 shows a locally variable coordinate frame 180, such as a Bishop frame, which may be employed in the techniques disclosed herein. Effectively, the image processing systems and methods operate by sequentially analyzing sub-volumes of a larger tissue volume, with the sub-volume representing a field of view as seen when flying along the already traced part of the elongate object center line in 3D. The Bishop curvature vector 185 may be an output of the AI model and may be integrated to determine new points along the center line.

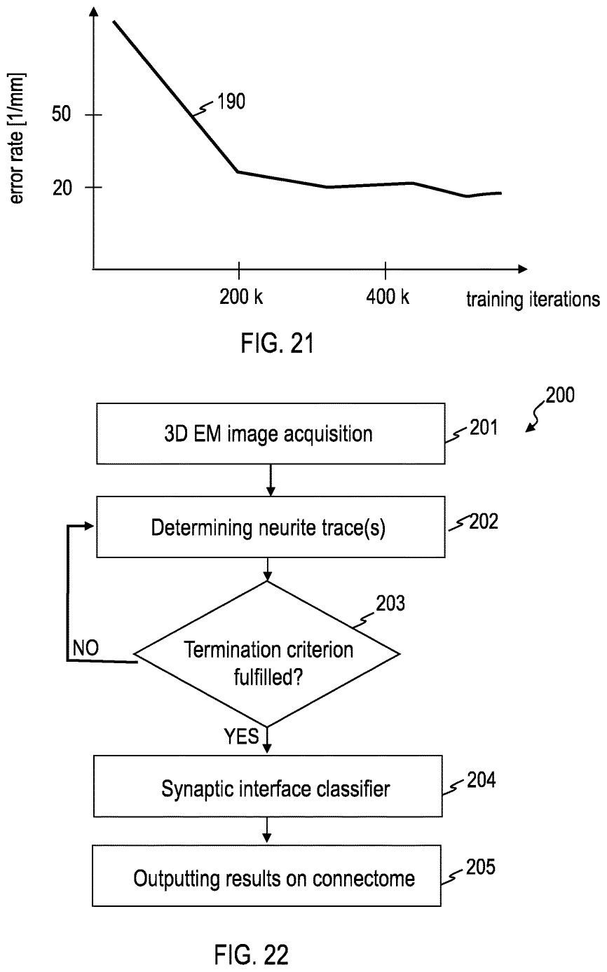

Techniques for training the AI model are also described in detail. As explained, training the AI model on positions and/or orientations that are off the center line provides enhanced stability. Error rates as low as about 14 errors/mm were attained even for complex tasks such as axon tracing or applications for spine necks, as described in association with the comparison to conventional techniques above. The error rate saturates with increasing number of training iterations, as shown by graph 190 in FIG. 21. In examples, about 700,000 training iterations were used herein for performance evaluation. Larger training sets may require more training iterations for saturation.

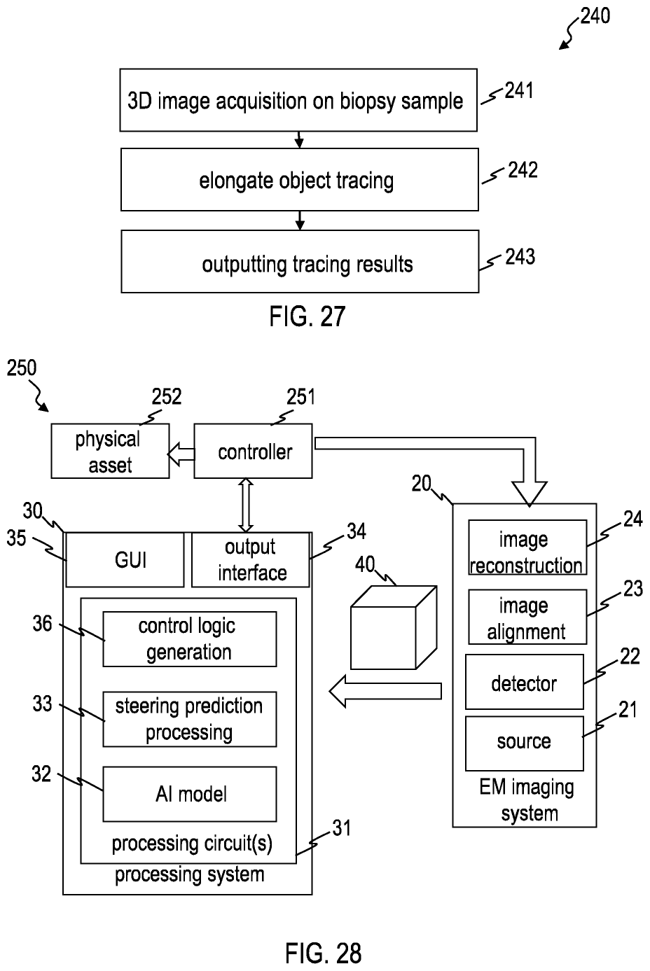

The image processing systems and methods disclosed herein may be used in various applications, such as connectome reconstruction (FIG. 22), analysis of biopsy samples (FIG. 27), and/or neuroscience-inspired artificial intelligence (FIG. 28).

FIG. 22 is a flow chart of a method 200. The method 200 may be performed by or using an image processing system as disclosed herein. At step 201, 3D EM image acquisition is performed. The 3D EM image acquisition may be performed on a bioptic sample. The bioptic sample is a sample that is not implanted back into a living animal or human after imaging.

At step 202, a trace of at least one neurite is determined. The traces of several neurites may be determined concurrently, e.g. by operating several instances of the image processing system in parallel. Additionally this step may comprise a more complex logic for iterative reconstruction and may contain the module 80 from FIG. 5 as a submodule.

At step 203, a termination criterion is checked. If the termination criterion is not fulfilled, the method returns to step 202. The method may be continued until all neurites of interest are traced.

In this manner, several neurites may be traced in the volume. Neurite tracing may comprise tracing axons, such as determining the axon connectome, and/or dendrites.

At step 204, synapse information is computed. This can be done using a synaptic interface classifier known to the skilled person without being limited thereto.

At step 205, when the termination criterion is fulfilled (e.g., when all neurites of interest have been traced), results may be output. The results may be output via a GUI or data interface, without being limited thereto.

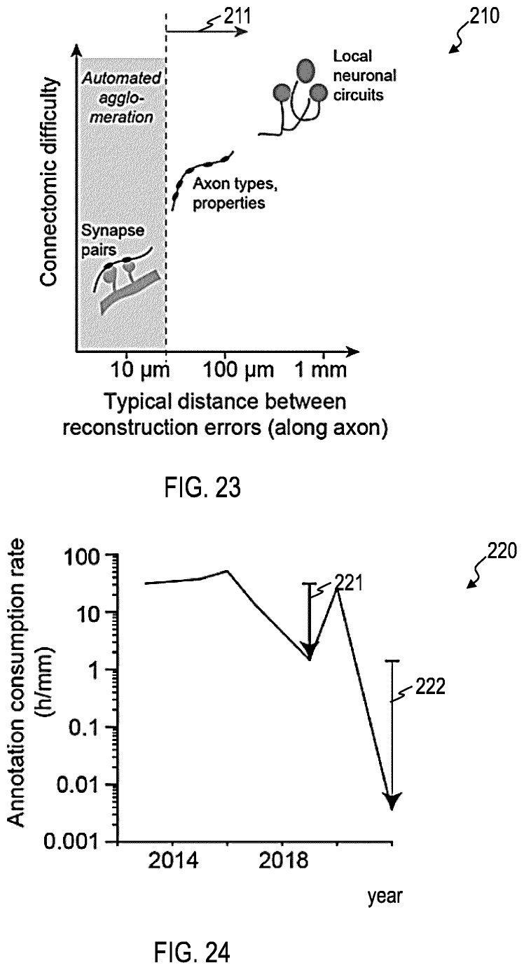

FIG. 23 is a graph 210 which depicts error rates required for various uses in association with connectomics analyses. The low error rates and large distances between successive errors attained by the methods and system of the invention, as illustrated by region 211, afford application of these techniques to use cases that were previously not readily addressed by conventional computerized techniques.

FIG. 24 is a graph 220 depicting an evolution of the time required for one mm of annotation, as a function of the techniques available in recent years. The techniques of Motta et al. 2019. “Dense Connectomic Reconstruction in Layer 4 of the Somatosensory Cortex.” Science. Vol 366. Issue 6469. p. eaay3134 provide an improvement 221. A further, very pronounced decrease 222 in the annotation consumption rate is provided by the methods and systems of the invention as disclosed herein.

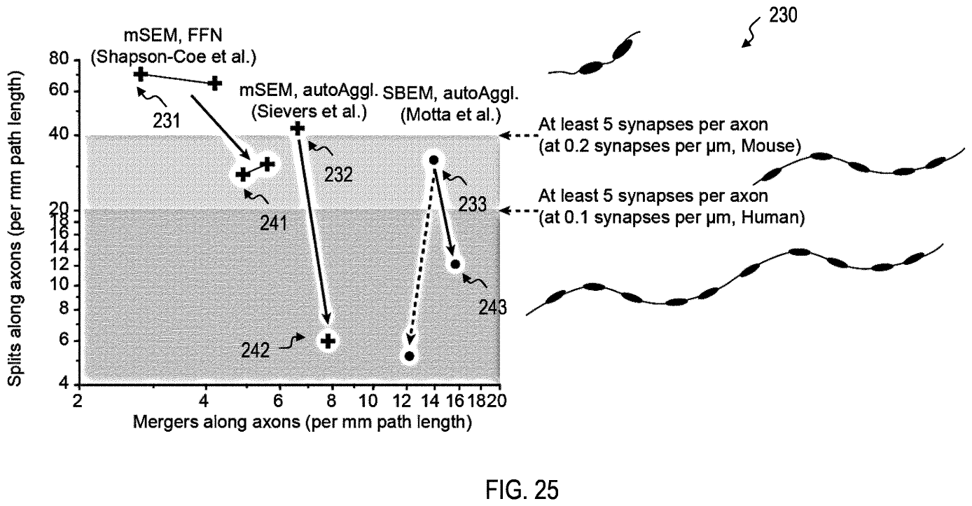

FIG. 25 is a graph 230 that quantifies a rate of axon splits and mergers, reported for randomly seeded axons. Results 231 were obtained for a FFN-based reconstruction of human cortex (A. Shapson-Coe et al. (2021). “A connectomic study of a petascale fragment of human cerebral cortex.” bioRxiv, 2021.2005.2029.446289. doi: 10.1101/2021.05.29.446289 (2021)). The techniques disclosed herein provide significantly improved results 241 for both segmentation states analyzed, with an increase in merger rate. Note that split rate requirements depend on synapse rate along axons: for at least 5 synapses per axon, split rates below 40 per mm and 20 per mm are required for typical mouse and human axons, respectively. Moving automation into these regimes is therefore desirable for more difficult connectomic analyses. The methods and systems disclosed herein improved densely seeded axon reconstruction for large-scale data from mouse cortex. The results 232 were obtained using a conventional technique (unpublished), as compared to the inventive techniques which provide improved results 242. For SBEM data, the methods and systems disclosed herein allow the full replacement of human annotation for the employed analyses, providing an improvement over the results 233 obtained using a conventional technique of Motta et al., loc. cit. The improved result obtained by the methods and systems disclosed herein is shown at 243. The potential added benefit of a further improvement by a factor 2 that might be attainable from human annotations (black dashed line) is not relevant for the analyses performed.

FIG. 26 is a further graph 260 that quantifies a rate of axon splits and mergers, reported for randomly seeded axons. For FIG. 26, the techniques were applied to 3D EM data obtained using a state-of-the-art high-throughput 3D EM imaging approach followed by multibeam scanning electron microscope (multiSEM). The data were obtained for both a mouse cortex and a human cortex. Data 261, 262, 263 correspond to conventional techniques. Data 264 corresponds to data obtained additionally using human annotations. Data 271, 272, 273 correspond to data obtained using the methods and systems disclosed herein.

After automated agglomeration, the methods and systems disclosed herein were seeded at automatically detected endings of axon agglomerates, and used to connect split axonal agglomerates. As a result, split rates of axons were reduced 7-fold (42.7 to 6.0 per mm axon path length) while only modestly increasing merger rates (3.3 to 4.5 per mm, as seen in FIG. 26, top datapoint 262 as compared to bottom datapoint 272). When directly comparing split rates at the same rate of merge errors (3.9 merge errors per mm axon path length) with a higher agglomeration, the agglomeration only solves 25% of split errors yielding 31.9 splits per mm, while the methods and systems disclosed herein solve 79% yielding 9.0 splits per mm. Hence, in this split/merge error regime the methods and systems disclosed herein improve split resolution 3.1-fold over standard agglomeration (FIG. 26, datapoints 262 versus top datapoint 272).

The methods and systems disclosed herein were evaluated on a subvolume of size (150 μm)³ from A. Shapson-Coe et al. (2021). “A connectomic study of a petascale fragment of human cerebral cortex.” bioRxiv, 2021.2005.2029.446289. doi: 10.1101/2021.05.29.446289 (2021) and found that application of the methods and systems disclosed herein on axonal endings obtained from the dense reconstructions of an FFN segmentation (M. Januszewski et al., “High-precision automated reconstruction of neurons with flood-filling networks.” Nat Methods. 2018 August; 15(8):605-610. doi: 10.1038/s41592-018-0049-4. Epub 2018 Jul. 16. PMID: 30013046) solves 57% of the splits on a random set of axons traced throughout the subvolume. This reduced the split rate from 65 splits/mm to 28 splits/mm, reaching split-lengths required for automated connectomic analyses while introducing few new merge errors (merge rate increased from 1.7 mergers/mm to 3.1 mergers/mm, as seen in FIG. 26, datapoint 261 (bottom) as compared to datapoint 271 (bottom)), which are tolerable merge rates for the kinds of connectomic analyses studied, e.g., in the context of the mouse cortex SBEM reconstructions. When comparing split error reduction at identical merge error rate (c2: 1.7 mergers/mm) for FFN and the methods and systems disclosed herein, it is found that the FFN-based agglomeration from c3 to c2 only solves 8% of the split errors of c3. In contrast, agglomeration based on the methods and systems disclosed herein solves 28% of split errors of c3 at identical merge error rate as c2 thereby being 3.5-fold more effective in split resolution than FFN (FIG. 26, datapoint 261 (top) as compared to 261 (bottom) and 271 (top)).

In more detail, FIG. 26 shows a quantification of a rate of axon splits and mergers, reported for randomly seeded axons. For flood-filling networks (FFN) based reconstruction of human cortex (A. Shapson-Coe et al. (2021). “A connectomic study of a petascale fragment of human cerebral cortex.” bioRxiv, 2021.2005.2029.446289. doi: 10.1101/2021.05.29.446289 (2021)), two agglomeration states were analyzed (c2, c3). A change in agglomeration parameters of FFN from c3 to c2 only solves 8% of c3 splits in dense axons (FFN datapoint 261), while application of the top 40% of validated tracings obtained using the methods and systems disclosed herein with highest certainty from c3 endings solves 28% of the split errors at the same merge error rate as c2 corresponding to a 3.5-fold improved split resolution (datapoint 271). With only a modest increase of 1.4 merge errors per mm the application of all validated tracings obtained using the methods and systems disclosed herein from c2 endings further reduces the split rate from 65 splits per mm to 28 splits per mm solving 57% of c2 split errors. In human cortex split rates below 30 splits per mm (approx. ≥30 μm axon fragments) are, e.g., required for an accurate distinction between excitatory and inhibitory axon fragments, see S. Loomba et al. (2022). “Connectomic comparison of mouse and human cortex.” Science. 2022 Jul. 8; 377(6602): eabo0924. doi: 10.1126/science.abo0924. Epub 2022 July 8. PMID: 35737810, while in mouse cortex at the same split rate, the associated ≥10 synapses per axon fragment, enable, e.g., the analysis of inhibitory target specificities (A. Motta et al. (2019). “Dense connectomic reconstruction in layer 4 of the somatosensory cortex.” Science: eaay3134). Moving automation into this regime is therefore critical for most connectomic analyses. For large-scale data from mouse cortex (Si150L4, accessible at https://wklink.org/7122), the methods and systems disclosed herein improve densely seeded axon reconstruction 7-fold, while only slightly increasing merge errors from 3.3 to 4.5 per mm. Taking the 90% agglomeration threshold as a reference, partial application of tracings obtained using the methods and systems disclosed herein yields the same merge error rate while reducing splits 3.1-fold more effectively than the agglomeration. For SBEM data, the methods and systems disclosed herein allow the full replacement of human annotation for the employed analyses (A. Motta et al. (2019). “Dense connectomic reconstruction in layer 4 of the somatosensory cortex.” Science: eaay3134). The added benefit of factor 2 from manual annotations (black dashed line to data 264) was not relevant for the analyses performed in that study. For fully automated analysis of local circuitry, error rates below 10 per mm are desirable. Such error rates can be attained using the techniques disclosed herein (data 272, 273).

For the evaluation of split and merge errors of agglomerations before and after error correction performed by the methods and systems disclosed herein (FIG. 26), merge errors were detected which extended further than 2.2 μm from the ground truth and manually verified that this heuristic accurately detects merge errors. For agglomerations prior to corrections performed by the methods and systems disclosed herein, each merge error was counted as ½ and divided by the ground truth path length to yield the merge error rate. This is because each merge error usually connects two neurites and counting them as 1 error per neurite instead of ½ would overestimate the total amount of merge errors. For sparse evaluations of error correction performed by the methods and systems disclosed herein limited to agglomerates that overlap with the ground truth, as done in both multiSEM datasets evaluated, additional mergers introduced by the methods and systems disclosed herein were counted as 1 instead of 0.5, which accounts for mergers from agglomerates not overlapping with ground truth and therefore not observable in a sparse evaluation. For split errors, the set of agglomerates was limited to be evaluated to those agglomerates that overlap more than 2.5 μm with the ground truth. This overlap threshold was introduced in order to avoid domination of split errors by many small agglomerates or unagglomerated segments along thin stretches of axons. Despite this overlap length threshold around 90% of the ground truth was still covered.

To test if a correction based on the methods and systems disclosed herein also allows for reduced split error rates when compared at the same merge error rate as the segmentation/agglomeration for the two multiSEM datasets, the following strategy was used to only apply subsets of tracings obtained using the methods and systems disclosed herein thereby limiting merge error rates: Prediction uncertainty using Monte-Carlo dropout was quantified for every step performed by the methods and systems disclosed herein as previously used to identify spine neck directions. Then a maximum uncertainty over steps was taken and forward and validation tracings were combined with a minimum of the two uncertainty scores. This approach is, thus, based on the concept to first score every direction of the validated tracings according to the position with maximum uncertainty and then, since the forward and backward tracings yielded the same flight path, the minimum of those two uncertainty scores was taken to quantify the uncertainty of the tracing as a whole. Application of validated tracings obtained using the methods and systems disclosed herein up to percentiles of 0.2, 0.4, 0.6, 0.8 of tracing uncertainties then yielded partial corrections performed by the methods and systems disclosed herein. These were evaluated for split and merge error rates and those with minimal split error rates at same merge error rate as segmentations/agglomerations were added as intermediate points within the split merge error plane of FIG. 26. Specifically, for the mouse cortex multiSEM dataset, application of the top 80% of validated tracings obtained using the methods and systems disclosed herein on the agglomeration at 85% agglomeration threshold yielded the same merge error rate as the agglomeration at 90% agglomeration threshold. Similarly, for the human cortex multiSEM dataset, the application of the top 40% of validated tracings obtained using the methods and systems disclosed herein on the FFN c3 agglomeration yielded the same merge error rate as the FFN c2 agglomeration (A. Shapson-Coe et al. (2021). “A connectomic study of a petascale fragment of human cerebral cortex.” bioRxiv, 2021.2005.2029.446289. doi: 10.1101/2021.05.29.446289 (2021)).

FIG. 27 is a flow chart of a method 240. The method 240 may be performed by or using an image processing system as disclosed herein. At step 241, 3D EM image acquisition is performed. The 3D EM image acquisition may be performed on a bioptic sample. The bioptic sample is a sample that is not implanted back into a living animal or human after imaging.

At step 241, image acquisition is performed on a biopsy sample. The image acquisition may be a 3D EM image acquisition on a bioptic sample that will be discarded after imaging.

At step 242, a trace of at least one neurite is determined. The traces of several neurites may be determined concurrently, e.g. by operating several instances of the image processing system in parallel.

At step 243, results of the tracing are output. The results may be output via a GUI (e.g., by outputting a visual representation of parts of a connectome via an optical output device) or data interface, without being limited thereto.

The image processing systems and methods disclosed herein may be used in the process of generating, training, and/or operating a processing logic (such as a signal processing logic) for controlling at least one physical asset (such as a machine).

FIG. 28 shows a system 250 which comprises an image processing system operative as disclosed herein. The image processing system may be operative to determine information on (parts of) a connectome and/or other structural information of the nervous system of a human or other animal.

The system 250 comprises a physical asset 252 (such as a machine) which is controlled by a controller 251. The controller 251 executes a machine learning, ML, model to process signal inputs (which may include sensor inputs from sensor measurements) and generates control signals that affect operation of the physical asset 252.

The ML model executed by the controller 251 may be generated by a control logic generation module 36 of the processing system 30. The control logic generation module 36 of the processing system 30 may use results of the connectome analysis to at least one of: (i) select or generate the ML model with a ML model architecture that depends on the connectome; (ii) train the ML model having the ML model architecture that depends on the connectome; and/or (iii) deploy the trained ML model that depends on the connectome as the signal processing logic to the controller 251 for execution. The controller 251 may execute the ML model that depends on the connectome determined by the processing system 30 for controlling the physical asset 252. The controller 251 may also feed back information to the processing system 30. For illustration, operation of the controller 251 and/or asset 252 may be monitored during field use. The processing system 30 may be caused to repeat and/or modify the object tracing during field use of the controller 251 and/or asset 252. In this way, the control logic may be updated during field use.

Alternatively or additionally, the controller 251 may be coupled to the image acquisition system 20 to control operation thereof. This may be done responsive to the results of the image processing performed by the image processing system 30.

### Techniques

Aspects of techniques that may be implemented in the image processing systems and methods will be discussed. It will be appreciated that alternative implementations may be used in the image processing systems and methods.

### EM Image Data Sets

The image processing systems and methods were trained and tested on a SBEM dataset from layer 4 primary somatosensory cortex of a 28 days old mouse. The tissue was conventionally en-bloc stained (K. L. Briggman et al. 2011. “Wiring Specificity in the Direction-Selectivity Circuit of the Retina.” Nature 471 (7337): 183-88) and imaged at 11.24×11.24 nm² and nominal cutting thickness of 28 nm. Oversegmentation was used to generate volume reconstructions of training set axons. The oversegmentation was obtained using the techniques of M. Berning et al. 2015. “SegEM: Efficient Image Analysis for High-Resolution Connectomics.” Neuron 87 (6): 1193-1206) with parameters as set for the whole-cell segmentation of the cortex dataset.

The image processing systems and methods were also trained and tested on a dataset from the barrel cortex of a mouse acquired with a multibeam scanning electron microscope. The tissue was stained following the protocol by Y. Hua et al. 2015. “Large-Volume En-Bloc 599 Staining for Electron Microscopy-Based Connectomics.” Nature Communications 6 (August): 7923 with small modifications and imaged at 4×4 nm² and nominal cutting thickness of 35 nm.

The image processing systems and methods were also evaluated on a multiSEM dataset with voxel size 4×4×33 nm³ (A. Shapson-Coe et al. (2021). “A connectomic study of a petascale fragment of human cerebral cortex.” bioRxiv, 2021.2005.2029.446289. doi: 10.1101/2021.05.29.446289 (2021)) segmented and agglomerated using Flood Filling Networks (M. Januszewski et al. 2018. “High-Precision Automated Reconstruction of Neurons with Flood-Filling Networks.” Nature Methods. https://doi.org/10.1038/s41592-018-0049-4).

### Skeleton Interpolation

Interpolation may be used to obtain a continuous representation and/or center line nodes with a desired step size of neurite branches (e.g. axonal branches) from sparsely placed nodes along the center axis.

The interpolation may be implemented as a 4th order B-spline interpolation (L. Piegl and W. Tiller. 1996. The NURBS Book. 2nd ed. Springer Science & Business Media.). For illustration, the curve may be parametrized to get a curvature adaptive step size ∥ [symbol] ∥Δt of

$$‖ \text{?} ‖ Δ t = f \cdot \frac{d}{1 + \frac{p}{2} κ} ,$$

where κ is the curvature, and ƒ is a step size factor, d is the default step size if the curvature is zero and ƒ=1, and p is the physical size of the projection plane. The default step size d can be selected to be equal to the smallest length of a voxel. This size may depend on the imaging system and may be, e.g., 11.24 nm for the specific SBEM dataset used in some examples herein.

The adaptive step size ensures that no voxel up to a radius of half the projection plane size in the projection plane at the current position of the CNN is skipped. The same adaptive step size may optionally be used during inference for the inventive technique disclosed herein to determine the step size of the integration step that determines subsequent positions and orientations.

### Bishop Frame

The Bishop frame (R. L. Bishop. 1975. “There Is More than One Way to Frame a Curve.” The American Mathematical Monthly 82 (3): 246-51) consists of three orthonormal vectors, namely the tangential vector to the curve (also termed flight vector) \vec{t}, and two vectors \vec{n₁} and \vec{n₂}. The evolution equations of the Bishop frame and the curve \vec{γ} read as follows:

$$\frac{d}{ds} \vec{γ} = \vec{t}$$

$$\frac{d}{ds} \vec{t} = {k}_{1} {\vec{n}}_{1} + {k}_{2} {\vec{n}}_{2} ≡ \vec{k}$$

$$\frac{d}{ds} {\vec{n}}_{1} = - {k}_{1} \vec{t}$$

$$\frac{d}{ds} {\vec{n}}_{2} = - {k}_{2} \vec{t} .$$

where s is the arc length of curve \vec{γ} and is used as a parameter that parameterizes the curve \vec{γ} such that |d\vec{γ}/ds∥=1 holds. Similar equations up to a factor can be formulated for an arbitrary parametrization of curve \vec{γ}. Here, k₁ and k₂ are the Bishop curvatures associated to the normal vectors \vec{n₁} and \vec{n₂}, respectively. In the techniques disclosed herein the Bishop curvatures correspond to the steering predictions of the AI model. The curvature vector is defined as \vec{k}=k₁\vec{n₁}+k₂\vec{n₂}. The curvature k of the curve \vec{γ} is

$$κ = \sqrt{{k}_{1}^{2} + {k}_{2}^{2}} .$$

The invariance of the curvature vector under certain operations (such as flips and rotations around the tangential vector applied to both normal vectors and corresponding transformations of the Bishop curvatures) are used in the techniques disclosed herein in association with the AI model. For illustration, online data augmentation may be performed. The flight direction can also be flipped, which corresponds to a time reversal symmetry. On the center line this time reversal and tangential vector flip does not affect the Bishop curvatures, while for the flight policy discussed below this symmetry can be broken.

The Bishop frame has weaker requirements for the curve \vec{γ} than other frames (such as the Frenet-Serret frame) often used as local coordinate system in differential geometry of parameterized curves. For illustration, for a parametrized curve in 3D Euclidean space, the Bishop frame does not require κ≠0. Initial conditions together with the Bishop curvatures uniquely define the center line curve \vec{γ}. This makes the Bishop curvatures particularly suitable as AI model outputs, as this allows one to reconstruct the curve by means of above evolution equations. As the Bishop frame also exists for parametrized curves in N-dimensional Euclidean space with N≥3, the same mathematical framework can be used and the techniques disclosed herein are similarly applicable to N-dimensional data. Additionally, parametrized curves in 2-dimensional Euclidean space can be considered a special case of parametrized curves in 3-dimensional Euclidean space, such that also here the described methods are applicable.

### Flight Policy

A flight policy usable during AI model training (for enforcing convergence back to the center line when applying the AI model to a position off the center line) can be derived by considering (a) the second order Taylor series expansions for the known center line curve \vec{γ}(s) with corresponding Bishop frame \vec{t}, \vec{n₁}, and \vec{n₂} and curvature vector \vec{k}, and (b) the second order Taylor series expansions for the unknown trajectory converging back to the center line with off-center line position \vec{γ}(s) and corresponding Bishop frame \vec{t}, \vec{n₁}, and \vec{n₂} and curvature vector \vec{n₁}:

$${\vec{γ}}_{T} ( s ) = \vec{γ} + s \vec{t} + \frac{{s}^{2}}{2} \vec{k}$$

$$\underline{{\vec{γ}}_{T}} ( s ) = \underline{\vec{γ}} + s \underline{\vec{t}} + \frac{{s}^{2}}{2} \underline{\vec{k}} .$$

For an off-center line position \vec{γ₁} and associated off direction \vec{t₁} as well as associated closest position on the axon center line \vec{γ} with corresponding center line aligned direction \vec{t} and curvature vector \vec{k}, convergence back to the on-center line tracing can be formulated as minimization of the future distance ∥\vec{Γ}_{T}∥ defined by:

$${\underbrace{{\vec{γ}}_{T} - \underline{{\vec{γ}}_{T}}}}_{{\vec{Γ}}_{T}} = {\underbrace{\vec{γ} - \underline{\vec{γ}}}}_{{Γ}^{→︀}} + s {\underbrace{( \vec{t} - \underline{\vec{t}} )}}_{{T}^{→︀}} + \frac{{s}^{2}}{2} {\underbrace{( \vec{k} - \underline{\vec{k}} )}}_{{K}^{→︀}} .$$

The optimization problem that minimizes ∥\vec{Γ}_{T}∥ within a distance (of the parameter s) of s_{c}, can be denoted as:

∇\vec{k}∥\vec{Γ}_{T}(s_{c})∥²=0,

where ∇_{\vec{k}}= [symbol] ∂ₖ_{₁}+ [symbol] ∂ₖ_{₂}.

Defining the projection operator

[symbol]

_{\vec{n}}_{₁}_{,\vec{n}}_{₂}=\vec{n}₁⊗\vec{n}₁+\vec{n}₂⊗\vec{n}₂

where ⊗ denotes an outer product, the solution of the minimization problem can be expressed as:

$$\underline{\vec{k}} = {𝒫}_{{\vec{\underline{n}}}_{1} , {\vec{\underline{n}}}_{2}} ( \frac{2}{{s}_{c}^{2}} [ \vec{Γ} + {s}_{c} \vec{t} ] + \vec{k} ) .$$

The parameter s_{c} is a free parameter of the flight policy. As discussed above, the parameter s_{c} may be chosen in dependence on distance of the current off-center line position from an obstacle, such as a membrane (e.g., a cell membrane). Thereby, an obstacle (e.g., membrane) avoiding technique can be implemented during training. As the above derivation is based on a Taylor series expansion, convergence to the center line is achieved through iterative application of above steering.

Direction Prediction with Monte-Carlo Dropout

The technique disclosed in this section can be used to, e.g., perform direction prediction for obtaining an initial steering prediction. A number N (e.g., N=256) equidistant or approximately equidistant orientations \vec{t}ᵢ on the surface of the unit sphere can be sampled. Monte-Carlo Dropout can be run with M (e.g., M=128) samples. From the sampled Bishop curvature predictions \vec{k}ᵢₘ, the mean curvatures [symbol] κ [symbol] ᵢ and the covariance covᵢ of the Bishop curvature can be calculated for each orientation. The uncertainty estimate for a direction can be determined as the square-root of the largest eigenvalue of the covariance matrix divided by the mean curvature,

uᵢ={square root over (max(eigvals(covᵢ)))}/ [symbol] κ [symbol] ᵢ

This quantity is similar to a coefficient of variation.

Using cosine similarity for neighboring angles within 30 degrees, a weighted average of uncertainties can be determined, e.g. as

$${\hat{u}}_{i} = \frac{{u}_{i}}{2} + \frac{1}{2} {\sum }_{j} {\hat{w}}_{ij} {u}_{i}$$

with weights

ŵᵢⱼ=wᵢⱼ/Σⱼwᵢⱼ

$${w}_{ij} = \{ \begin{matrix}〈 {\vec{t}}_{i} \vert {\vec{t}}_{j} {〉}^{16} & if arccos ( 〈 {\vec{t}}_{i} \vert {\vec{t}}_{j} 〉 ) < 30 ° \\ 0 & otherwise\end{matrix}$$

The weighted averaging provides enhanced stability.

The orientation with minimal averaged uncertainty may be used as the first orientation candidate.

For spine heads, a second candidate may be chosen which is at least 110° from the first candidate and has a minimal averaged uncertainty from among the remaining orientations.

### Local Realignment

As was already discussed, a local realignment may be performed in the method and/or by the imaging system 20. The local realignment may be performed by image alignment component 23 or the imaging system 20 may be operative to execute a second, local image realignment component in addition to the image alignment component 23.

The local realignment techniques will be described, at least in part, in association with 3D EM data. It should be understood that these techniques may also be used in association with other 3D image data or sets of 2D image data.

The local realignment may comprise the following steps executed to perform a local realignment of slices of image data along a slicing direction: determining slice-to-slice shift vectors for a plurality of slices along the slicing direction; identifying valid slice-to-slice shift vectors between slices; using the valid slice-to-slice shift vectors to generate a locally realigned subvolume.

The locally realigned subvolume may be fed to the AI model input, e.g., to an input layer of a CNN or RNN or to a filter or other preprocessing that preprocesses the realigned subvolume prior to it being processed by the CNN or RNN.

Determining the slice-to-slice shift vectors may comprise determining an auto- and cross-correlations of slices.

Determining the slice-to-slice shift vectors and/or their validity may comprise determining a mean and a standard deviation of correlation-weighted shift vectors based on a first threshold criterion.

Identifying valid slice-to-slice shift vectors may comprise verifying that the determined auto-correlation fulfills a first criterion (such as having a peak at a desired shift, e.g., at zero shift).

Identifying valid slice-to-slice shift vectors may comprise verifying that the determined auto-correlation peak has a standard deviation that fulfills a second criterion (such as having a standard deviation that fulfils a second threshold criterion, e.g., being less than or equal to a certain number of pixels).

Identifying valid slice-to-slice shift vectors may comprise verifying that a combination of two successive auto- and/or cross-correlation peaks fulfils a third criterion (e.g., fulfils a third threshold criterion, where the third threshold criterion may optionally be dependent on surrounding auto- and/or cross-correlations).

Identifying valid slice-to-slice shift vectors may comprise verifying that the determined cross-correlation fulfills a first cross-correlation criterion that depends on auto-correlations for the two involved slices (such as the mentioned auto-correlation criteria being valid for both slices).

Identifying valid slice-to-slice shift vectors may comprise verifying that the determined cross-correlation normalized with the expected cross-correlation value for fully correlated intensities fulfills a second cross-correlation criterion (such as a peak of the cross-correlation normalized with the expected cross-correlation value for fully correlated intensities fulfilling yet another threshold criterion, such as being equal to or greater than a fourth threshold).

Identifying valid slice-to-slice shift vectors may comprise verifying that a standard deviation of the shift vector fulfills a third cross-correlation criterion (such as the standard deviation being equal to or less than a fifth threshold).

Using the valid shift vectors may comprise identifying one or several slices to be realigned even though the slice-to-slice shift vector is not considered valid, and computing the shift vector in this case from valid shift vectors relating to a neighborhood of these one or several slices.

Using the valid shift vectors may comprise generating a locally realigned and trilinearly interpolated subvolume.

Generating the locally realigned and trilinearly interpolated subvolume may comprise performing floor or ceil operations.

Generating the locally realigned and trilinearly interpolated subvolume may comprise performing a trilinear interpolation as a sequence of linear interpolations along cardinal axes.

The sequence of linear interpolations may comprise four linear interpolations along a first dimension, two linear interpolations along a second dimension, and one linear interpolation along a third dimension.

The locally realigned subvolume may be processed to trace an elongate object, such as a blood vessel or neurite, in 3D.

To efficiently reconstruct neurites across discontinuities in the globally aligned 3D image data, the methods and systems disclosed herein may be operative to apply an alignment method locally on a potentially imperfect global alignment, followed by a neurite reconstruction method receiving the locally realigned 3D subvolume, similar to the techniques of P. H. Li et al., “Automated Reconstruction of a Serial-Section EM Drosophila Brain with Flood-Filling Networks and Local Realignment.” Preprint. https://doi.org/10.1101/605634. The reconstructions within the subvolume then need to be dealigned, i.e., projected back into a global reference frame (as discussed in the cited document by P. H. Li et al.).

The following discussion is focused on local realignment of 2D slices across the slicing axis ignoring problems stemming from in-plane stitching of 2D mosaics with overlapping fields of view. Trading-off accuracy and efficiency, the slice-to-slice shift vectors for local realignment are precomputed on a grid for a whole dataset and bilinearly interpolated to yield approximate per-voxel localized alignment, where the size of the grid for precomputed shift vectors is a hyperparameter of the local realignment method.

To precompute slice-to-slice shift vectors for local realignment, 2D image data from slices zᵢ of N×N grayscale EM data at 8×8 nm² resolution with, e.g., N=512 and pixel values in a range [0, 255] may be mean-subtracted and divided by 255. Then, locations of global maxima of auto/cross-correlations of slices (zᵢ, zᵢ), (zᵢ, zᵢ₊₁) and (zᵢ, zᵢ₊₂) may be determined corresponding to pixel-level shift vectors \vec{s}_{i,i}, \vec{s}_{i,i+1}, \vec{s}_{i,i+2}. In addition, the mean and standard deviation of correlation-weighted shift vectors reaching a correlation of at least two-thirds of the maximum correlation relative to the noise level are computed. Specifically, shift vectors were considered if the correlation exceeded σ_{i,j}+⅔(max(π_{i,j},σ_{i,j})−σ_{i,j}), where the peak cross-correlation between slices i, j is π_{i,j}, and the noise level σ_{i,j}=\sqrt{N²}std(zᵢ)std(zⱼ) (i.e., standard error of the correlation expected under the assumption of independently and identically distributed pixel intensity values within each slice).

To filter irregular shift-vectors, a number of criteria can be used. For illustration, the realignment may be implemented such that, for the auto-correlation of slice zᵢ to be valid, the following conditions must apply: (1) auto-correlation must peak at zero shift, i.e., \vec{s}_{i,i}=(0, 0), and (2) must be sharp enough with a standard deviation of ≤2 pixels, and (3) the peak cross-correlation π_{i,j} normalized with the expected cross-correlation value e_{i,j}=N²std(zᵢ)std(zⱼ) for fully correlated intensity values of two successive (zᵢ₋₁,zᵢ) and (zᵢ,zᵢ₊₁) cross-correlations subtracted from the same metric of surrounding three cross-correlations, (zᵢ₋₃,zᵢ₋₂), (zᵢ₋₂,zᵢ₋₁) and (zᵢ₊₁,zᵢ₊₂), must be ≥−0.095.

The realignment may be implemented such that, for shift vectors \vec{s}_{i,i+1} from the cross-correlation of slices (zᵢ,zᵢ₊₁) to be valid, the following must apply: (1) auto-correlation conditions must be true for both slices, (2) the peak cross-correlation normalized with its respective maximum must be ≥0.18, (3) the shift vector standard deviation must be ≤10 pixels.

The realignment may be implemented such that, for locations, for which \vec{s}_{i,i+1} are invalid (in accordance with the above-described criterion for shift vectors to be valid), shift vectors \vec{s}_{i,i+2} from (zᵢ,zᵢ₊₂) slices are considered if the following criteria are met: (1) auto-correlation conditions (see above) must be true for both slices and (2) the shift vector standard deviation must be ≤11 pixels.

The realignment may be implemented such that, to compute otherwise invalid shift vectors \vec{s}_{i,i+1}, valid shift vectors \vec{s}_{i,i+2} and/or \vec{s}_{i-1,i+1} may be used. Setting i=2 without loss of generality, given valid shift vectors \vec{s}_{1,2} and \vec{s}_{3,4}, and at least one valid shift vector \vec{s}_{1,3} and/or \vec{s}_{2,4}, the invalid shift vector \vec{s}_{2,3} is computed as \vec{s}_{2,3}=\vec{s}_{1,3}−\vec{s}_{1,2}, or \vec{s}_{2,3}=\vec{s}_{2,4}−\vec{s}_{3,4}, or as the average of those two if both \vec{s}_{1,3} and \vec{s}_{2,4} are valid. Similarly, given valid shift vectors \vec{s}_{1,2},\vec{s}_{4,5}, and \vec{s}_{2,4}, the invalid shift vectors \vec{s}_{2,3} and \vec{s}_{3,4} are approximated as \vec{s}_{2,3}=\vec{s}_{3,4}=_{0.5}·\vec{s}_{2,4}. Remaining invalid shift vectors \vec{s}_{i,i+1} are set to \vec{s}_{i,i+1}=(0, 0).

Shift vectors s_{i,i+1} as determined above may be precomputed for every z-slice on a regular grid with a grid spacing of 512 pixels along in-plane x-y axes. To approximate localized shift vectors for any position in the global reference frame, bilinear interpolation may be employed. To generate a locally realigned and trilinearly interpolated subvolume that can be fed to the AI model (e.g., to the CNN or another AI model input), the current position v may be taken as the reference to compute local shift vectors such that a slice at z=└(\vec{γ})_{z}/s_{z}┘ has a shift of 0, where s_{z}=35 nm is a voxel size along the slice axis and └x┘ refers to a floor operation. To this end, first, the shift vectors of neighboring slices for x/y locations (\vec{γ})ₓ/sₓ and (\vec{γ})_{y}/s_{y} may be determined using bilinear interpolation of all relevant slice-to-slice shift vectors \vec{s}_{i,i+1} for this subvolume. Then, a cumulative sum may be used to aggregate slice-to-slice shift vectors across z coordinates of neighboring slices and from the result the aggregated shift vector of slice └(\vec{γ})_{z}/s_{z}┘ may be subtracted. This yields shift vectors with respect to the current position \vec{γ} and hence a shift of 0 at slice └(\vec{γ})_{z}/s_{z}┘.

To trilinearly interpolate taking shift vectors into account, the method and system may make use of the formulation of a trilinear interpolation as a sequence of linear interpolations along cardinal axes: (1) four linear interpolations along a first dimension, (2) two linear interpolations of results from (1) along a second dimension and (3) one linear interpolation of the results from (2) along a third dimension. Specifically, the interpolation of a single voxel can be computed by first linearly interpolating along x and y axes independently for floored and ceiled z values respectively with corresponding shifts along x and y applied, and then linearly interpolating along the z axis.

As predictions from the CNN on the locally realigned subvolume are only valid within this subvolume, they need to be dealigned to be related to the global coordinate system of a 3D EM dataset. The methods and systems disclosed herein may be operative to implement the dealignment, comprising first integrating the predicted Bishop curvatures to \vec{γ}ₙₑₓₜ as before and then applying the aggregated shift vectors from the start z slice └(\vec{γ})_{z}/s_{z}┘, used as reference for the local realignment of the subvolume, to a target z slice └(\vec{γ}ₙₑₓₜ)_{z}/s_{z}┘ onto \vec{γ}ₙₑₓₜ to yield a position within the global coordinate system. Unit vectors spanning the Bishop frame may be left unchanged in this process.

Alignment techniques may be used in association with reassembling of 2D images into a consistent 3D image volume with a common global reference frame. This is referred to as alignment. In case of errors in any of the preparation, slicing, imaging or reassembling steps, the resulting 3D image dataset will generally not be consistent at all locations. The realignment techniques disclosed herein are technically advantages for many imaging techniques disclosed herein. For illustration, to image neural tissue using 3D EM, the neural tissue is physically disassembled (sliced) along one axis, 2D-imaged with an electron microscope, potentially as a mosaic with overlapping fields of view, and virtually reassembled to ideally yield a globally consistent 3D image dataset. For this reassembly and/or after a coarse global reassembly, the realignment techniques disclosed herein may be applied. The alignment techniques disclosed herein provide good consistency while avoiding excessive computational runtimes. The alignment techniques disclosed herein are beneficial for many of the applications considered herein.

While embodiments have been described in detail, various modifications and alterations may be implemented in other embodiments.

For illustration, the techniques disclosed herein may be applied to tracing a wide variety of elongate objects.

The techniques disclosed herein are not limited to tracing elongate objects and/or to tracing in 3D but may be applied to a wide variety of other use cases, such as tracing an object through a series of image frames (e.g., in a video), tracing an object in a spatio-temporal coordinate system, and/or performing image processing for a wide variety of control actions.

1. A method of tracing an elongate object (41) in image data (40; 170), the image data (40; 170) being biological or medical image data (40; 170), the method comprising the following steps performed by an image processing system (30):
determining positions of a series of points (60) along the elongate object (41) in three-dimensional, 3D, space to trace the elongate object (41) in 3D in the image data (40; 170), wherein determining the positions of the series of points (60) comprises an iterative process, the iterative process comprising:
inferring, using an artificial intelligence, AI, model (110; 130), a steering prediction that is dependent on a direction along which the elongate object (41), optionally a center line (50) of the elongate object, extends from a previously determined point along the elongate object (41), the AI model (110; 130) comprising an input layer operative to receive an AI model input that is based on pixel or voxel values of the image data (40; 170) and an output layer operative to provide, in each iteration of the iterative process, an AI model (110; 130) output that is or indicates the steering prediction;
computing, using at least the steering prediction, a next point (62-65) of the series of points (60), the next point following the previously determined point (61-64) in the series of points (60); and
iteratively repeating the inferring and computing steps until a termination criterion is fulfilled, to thereby determine the positions of the series of points (60).

2. The method of claim 1,
wherein the AI model comprises
a convolutional neural network, CNN, comprising the input layer, a plurality of convolutional layers, a dropout layer, and the output layer, or
a recurrent neural network, RNN;
wherein the AI model input that is input to the input layer in an iteration of the iterative process represent a sub-volume (91, 93; 172-174) of a tissue volume represented by the image data (40; 170), wherein the AI model input
comprises pixel or voxel values representing the sub-volume of the image data (40; 170), or
is obtained by multivariate interpolation of pixel or voxel values representing the sub-volume of the image data (40; 170); and
wherein an orientation of the sub-volume (91, 93; 172-174) is dependent on the steering prediction determined in the preceding iteration of the iterative process.

3. The method of claim 2, wherein the steering prediction is dependent on a second derivative of a center line (50) of the elongate object (41)

4. The method of claim 1,
wherein the AI model is
a convolutional neural network, CNN, comprising the input layer, a plurality of convolutional layers, a dropout layer, and the output layer, or
a recurrent neural network, RNN;
wherein the AI model input that is input to the input layer in an iteration of the iterative process represent a sub-volume (91, 93; 172-174) of a tissue volume represented by the image data (40; 170);
the steering prediction being curvature values relative to a reference frame given in relation to the sub-volume such that it yields a position offset for determining in an iterative manner a series of points located on a center line (50) of the elongate object; and
in each iteration following an immediately preceding iteration, the respective subvolume that is processed is determined such as to have a rotational orientation that is determined by the direction of extension of the elongate object as determined from the steering prediction obtained in the immediately preceding iteration.

5. The method of claim 1, wherein one or several of the following applies:
determining the next point of the series of points (60) comprises integrating the steering prediction;
determining the next point of the series of points (60) comprises integrating the steering prediction, wherein integrating the steering prediction comprises integrating curvatures in a Bishop reference frame;
the steering prediction is related to a second derivative of the center line (50) of the elongate object (41).

6. The method of claim 1 or claim 5, wherein the AI model input that is input to the input layer in an iteration of the iterative process represent a sub-volume (91, 93; 172-174) of a tissue volume represented by the image data (40; 170), the sub-volume (91, 93; 172-174) being dependent on
the position and/or orientation and/or steering prediction determined in one or several preceding iterations of the iterative process.

7. The method of claim 6,
wherein an orientation of the sub-volume (91, 93; 172-174) is dependent on the preceding steering prediction.

8. The method of claim 6 or claim 7, wherein one of the following applies:
a center of the sub-volume (91, 93; 172-174) is shifted relative to the previously determined point by an offset;
a center of the sub-volume (91, 93; 172-174) is shifted relative to the previously determined point by an offset, wherein the offset is dependent on the steering prediction determined in the preceding iteration.

9. The method of any one of claims 6 to 8, further comprising a multivariate interpolation of pixel or voxel values of the image data (40; 170) to determine the AI model input.

10. The method of claim 9, wherein the multivariate interpolation comprises
weighted averaging of the pixel or voxel values to generate the AI model input as interpolated pixel or voxel values on a grid that is tilted and/or offset relative to a pixel or voxel grid of the image data (40; 170), or
trilinear projection.

11. The method of any one of the preceding claims, further comprising performing an initialization of the iterative procedure, wherein the initialization is dependent on whether an initial direction is available.

12. The method of claim 11,
wherein when the initial direction is not available, the method comprises determining the initial direction for use in an initial iteration of the iterative process.

13. The method of claim 12 wherein determining the initial direction comprises:
determining uncertainty estimates of predictions for various directions and
choosing the direction and steering prediction from among the various directions for which the uncertainty estimate in the corresponding prediction is minimum.

14. The method of claim 13,
wherein the uncertainty estimates are determined using Monte-Carlo dropout.

15. The method of any one of the preceding claims, wherein one of the following applies:
wherein the AI model (110; 130) comprises a convolutional neural network, CNN, or a recurrent neural network, RNN,
wherein the AI model (110; 130) comprises the input layer, a plurality of convolutional layers, a dropout layer, and the output layer,
wherein the AI model (110; 130) comprises one or several of pooling layers, attention modules, self-attention modules, atrous convolutions, normalization layers, batch normalization layers, transformers, recurrent layers.

16. The method of any one of the preceding claims,
wherein one, several, or all of the following applies or apply:
the output layer is a linear layer;
the plurality of convolutional layers comprises one or several strided convolutional layers;
the AI model (110; 130) is or comprises a fully convolutional network;
the AI model (110; 130) comprises one or several nonlinearities;
the AI model (110; 130) comprises exponential linear units, ELU;
the AI model (110; 130) comprises rectified linear units, ReLU;
the AI model (110; 130) comprises Scaled Exponential Linear units, SELU;
the AI model (110; 130) comprises Gaussian Error Linear Units, GELU.

17. The method of any one of the preceding claims, wherein the elongate object (41) is a neurite.

18. The method of any one of claims 1 to 16, wherein the elongate object (41) is a blood vessel.

19. The method of any one of the preceding claims, wherein the elongate object (41) has a surface that extends around the center line (50) in a locally cylindrical manner.

20. The method of any one of the preceding claims, further comprising training the AI model (110; 130).

21. The method of claim 20, wherein one or both of the following applies:
the AI model (110; 130) is trained using supervised learning;
training the AI model (110; 130) comprises gradient-based updates of AI model (110; 130) parameters.

22. The method of claim 20 or claim 21, wherein training the AI model (110; 130) comprises training the AI model (110; 130) to comply with a flight policy that causes the iterative process to converge to the elongate object (41) when starting from or reaching a location in the image data (40; 170) that is off the elongate object (41).

23. The method of claim 22, wherein a convergence distance of the flight policy determines how rapidly the iterative process converges back to the elongate object (41).

24. The method of claim 23, wherein one or both of the following applies:
the convergence distance is a dynamic parameter that is varied during AI model (110; 130) training and/or inference;
the convergence distance is set as a function of a distance from a physiological boundary during AI model (110; 130) training.

25. The method of claim 24, wherein the convergence distance is set as a function of a distance from a physiological boundary during AI model (110; 130) training, wherein the convergence distance is a monotonously increasing function of the distance from the physiological boundary.

26. A medical image processing method, comprising:
receiving medical image data (40; 170) that comprises a 3D image or a set of 2D images of tissue comprising neural tissue;
performing the method of any one of the preceding claims to trace an elongate object (41) in the medical image data (40; 170); and
outputting one or several of:
a visual representation of the series of points (60) or of the center line (50);
a machine-readable representation of the series of points (60) or of the center line (50);
a control signal that is dependent on the series of points (60) located along the center line (50);
a machine learning, ML, model (110; 130) that has an architecture that depends on the series of points (60) located along the center line (50).

27. A connectomics method, comprising performing the elongate object (41) tracing method of any one of claims 1 to 25.

28. The connectomics method of claim 27, further comprising executing a synaptic interface classifier and using at least an output of the elongate object tracing method and of the synaptic interface classifier to determine a connectome.

29. A method of analyzing a bioptic sample, comprising
acquiring image data (40; 170) of the bioptic sample;
processing the image data (40; 170) using the method of any one of claims 1 to 25; and
outputting a result of the image data (40; 170) processing via an interface.

30. A method of generating a signal processing logic for controlling a physical asset, the method comprising:
performing the elongate object tracing method of any one of claims 1 to 25;
using, by at least one computing system, an output of the elongate object tracing method to
select or generate a machine learning, ML, model (110; 130) architecture;
train an ML model (110; 130) having the ML model (110; 130) architecture;
deploy the trained ML model (110; 130) as the signal processing logic to a control device for execution;
control an image acquisition system (20) that acquires the image data (40; 170).

31. An image processing system (30) for tracing an elongate object (41) in image data (40; 170), the image processing system (30) comprising:
an interface operative to receive the image data (40; 170), the image data (40; 170) being biological or medical image data (40; 170), and
at least one processing device operative to:
determine positions of a series of points (60) along an elongate object (41) of the elongate object (41) in three-dimensional, 3D, space to trace the elongate object (41) in 3D in the image data (40; 170), wherein determining the positions of the series of points (60) comprises an iterative process, the iterative process comprising:
inferring, using an artificial intelligence, AI, model (110; 130), a steering prediction that is dependent on a direction along which the elongate object (41), optionally a center line (50) of the elongate object (41), extends from a previously determined point along the elongate object (41), the AI model (110; 130) comprising an input layer operative to receive an AI model input that is based on pixel or voxel values of the image data (40; 170) and an output layer operative to provide, in each iteration of the iterative process, an AI model (110; 130) output that is or indicates the steering prediction;
computing, using at least the steering prediction, a next point of the series of points (60), the next point following the previously determined point in the series of points (60); and
iteratively repeating the inferring and computing steps until a termination criterion is fulfilled, to thereby determine the positions of the series of points (60).

32. The image processing system (30) of claim 30, wherein the image processing system is operative such that:
the AI model comprises
a convolutional neural network, CNN, comprising the input layer, a plurality of convolutional layers, a dropout layer, and the output layer, or
a recurrent neural network, RNN;
the AI model input that is input to the input layer in an iteration of the iterative process represent a sub-volume (91, 93; 172-174) of a tissue volume represented by the image data (40; 170), wherein the AI model input
comprises pixel or voxel values representing the sub-volume of the image data (40; 170), or
is obtained by multivariate interpolation of pixel or voxel values representing the sub-volume of the image data (40; 170);
an orientation of the sub-volume (91, 93; 172-174) is dependent on the steering prediction determined in a preceding iteration of the iterative process.

33. The image processing system (30) of claim 32, wherein the image processing system is operative such that the steering prediction is dependent on a second derivative of a center line (50) of the elongate object (41).

34. The image processing system (30) of claim 31, wherein the image processing system is operative such that:
the AI model is
a convolutional neural network, CNN, comprising the input layer, a plurality of convolutional layers, a dropout layer, and the output layer, or
a recurrent neural network, RNN;
the AI model input that is input to the input layer in an iteration of the iterative process represent a sub-volume (91, 93; 172-174) of a tissue volume represented by the image data (40; 170);
the steering prediction being curvature values relative to a reference frame given in relation to the sub-volume such that it yields a position offset for determining in an iterative manner a series of points located on a center line (50) of the elongate object; and
in each iteration following an immediately preceding iteration, the respective subvolume that is processed is determined such as to have a rotational orientation that is determined by the direction of extension of the elongate object as determined from the steering prediction obtained in the immediately preceding iteration.
